(19) United States
(12) Patent Application Publication
(10) Pub. No.: US 2005/0065081 A1
(43) Pub. Date: Mar. 24, 2005

(54) Tyrosine kinase inhibitors

(76) Inventors: Karel Dorey, London (GB); Wilhelmina Pluk, Cuyk (NL); Giulio Superti-Furga, Heidelberg (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/501,891

(22) PCT Filed: Jan. 21, 2003
(86) PCT No.: PCT/GB03/00211

(30) Foreign Application Priority Data
Jan. 22, 2002 (GB) 0201384.5

(51) Int. Cl. C12N 9/99; A61K 38/52

(52) U.S. Cl. 514/12; 435/184

(57) ABSTRACT

The present invention relates to novel proteins that inhibit the activity of tyrosine kinases. In particular, the invention provides a tyrosine kinase inhibitor protein consisting of the cap region of a c-Abl protein. The invention also relates to the use of tyrosine kinase inhibitor proteins in the treatment and diagnosis of diseases, in particular cancers, in humans.

The present invention relates to novel proteins that inhibit the activity of tyrosine kinases. The invention also relates to the use of tyrosine kinase inhibitor proteins in the treatment and diagnosis of diseases, in particular cancers, in humans.

All documents mentioned in the text and listed at the end of the description are incorporated herein by reference.

Protein tyrosine kinases are enzymes that transfer the terminal phosphate of adenosine triphosphate (ATP) to a specific tyrosine residue on a target protein. These enzymes are found in all multicellular organisms and play a central role in the regulation of cellular growth and in the differentiation of complex eukaryotes.

There are two major classes of tyrosine kinases: transmembrane receptor tyrosine kinases and non-receptor tyrosine kinases. Regulation of all protein tyrosine kinases is essential for normal cellular differentiation and proliferation. While controlled activation of tyrosine kinases promotes normal proliferation, deregulated tyrosine kinases can cause neoplastic transformation. Examples from both classes of kinases have been shown to function as dominant oncogenes, generally as a result of overexpression and/or structural alteration.

Transmembrane receptor tyrosine kinases are activated directly by binding of peptide growth factors and cytokines to their extracellular domains. Tyrosine kinases which fall within this class include receptors for platelet-derived growth factor, fibroblast growth factors, hepatocyte growth factor, insulin, insulin-like growth factor-1, nerve growth factor, vascular endothelial growth factor and macrophage colony stimulating factor. The normal function of these receptors is to act as transducers of extracellular signals.

Some non-receptor tyrosine kinases are associated with cell surface receptors which do not have intrinsic tyrosine kinase activity. For example, members of the Src family of non-receptor protein tyrosine kinases in mammals (such as src, yes, fgr, fyn, lck, lyn, hck and blk) are mostly located on the cytoplasmic side of the plasma membrane, held there partly by their interaction with transmembrane receptors and partly by covalently-attached lipid chains. These proteins are also involved in signal transduction pathways. However, not all non-receptor protein tyrosine kinases are associated with transmembrane receptors. Some are found in the cytoplasm or even in the nucleus of cells. The role of these proteins is in many cases unknown.

The c-Abl protein tyrosine kinase is another example of a non-receptor tyrosine kinase. It was originally isolated as a cellular homologue of the v-abl oncogene of a transforming retrovirus, the Abelson murine leukaemia virus. The c-Abl protein is now known to be ubiquitously expressed and highly conserved in metazoan evolution. The N-terminal domain of the c-Abl protein, and the product of its paralog gene, Arg (ABL2), resemble Sic family kinases and consist of an SH3 domain, an SH2 domain and a catalytic domain (reviewed in Superti-Purga and Courtneidge, 1995; Van Etten, 1999). However, in contrast to Src family kinases, c-Abl and Arg lack a short C-terminal regulatory-tail and instead have a large C-terminal portion, encoded by a single exon and thus called the “last exon region”.

Although c-Abl was first identified 20 years ago, its physiological role is still unclear. It has been implicated in a wide range of cellular processes including cell differentiation, cell division, cell adhesion, cell death and stress response (Van Etten, 1999; Wang, 2000). It is found in both the nucleus and cytoplasm of cells and is thought to shuttle between the two subcellular departments (Taagepera et al., 1998). When expressed transiently in tissue culture cells, the ability of c-Abl to become phosphorylated on tyrosine and phosphorylate cellular substrates is considerably weaker than that of the natural oncogenic form BCR-Abl or of c-Abl's viral counterpart, v-Abl, suggesting that its kinase activity is tightly regulated.

In humans, chronic myelogenous leukemia (CML) and a subset of acute lymphocytic leukemia (ALL) are causally linked to the presence of the Philadelphia chromosome, which is the result of a translocation between chromosome 22 and chromosome 9. In this translocation, sequences of the first exon of the c-Abl tyrosine kinase gene (ABL1) are replaced by sequences from the BCR gene. Depending on the breakpoint in the BCR gene, the resulting fusion protein, BCR-Abl, can have molecular masses of 210, 190 (the two major forms) or 230 kDa The consequences of BCR-Abl on signal transduction pathways and the cellular effects have been studied extensively (Raitano et al., 1997). Dependent on the cell type, BCR-Abl expression results in enhanced proliferation, morphological transformation, or abrogation of growth factor or adhesion dependence. In general, the effects are growth stimulatory and anti-apoptotic. When forced into the nuclei of cells, however, BCR-Abl induces apoptosis (Vigneri and Wang, 2001).

The common feature critical for all the biological effects of BCR-Abl is its constitutively high level of tyrosine kinase activity derived from the catalytic domain in its ABL1 moiety. A small molecular inhibitor of BCR-Abl catalytic activity, STI571, has been described which binds to the ATP-binding pocket of the catalytic domain, but also interacts with less conserved regulatory structural elements, affecting their function (Schindler et al., 2000). STI571 appears to target an Abl-specific inactive conformation of the catalytic domain, explaining its high selectivity for Abl over other tyrosine kinases. Although STI571 initially appeared to be a promising therapeutic agent in clinical trials (Druker et al., 1996; Thiesing et al., 2000), it has recently been found that patients treated with ST1517 relapse because of a secondary mutation in the ATP binding pocket (Gorre et al, 2001).

There is therefore a need for improved drugs capable of inhibiting BCR-Abl. Part of the difficulty in developing improved drugs to target oncogenic forms of Abl is due to the fact that despite years of investigation, the molecular mechanism responsible for regulation of the c-Abl tyrosine linase has remained elusive. In particular, the mechanism responsible for natural inhibition of the cellular form of the enzyme is unknown.

Mutations have been identified in c-Abl, typically in the SH3 domain, that unleash the catalytic activity and often the oncogenic potential of the Abl protein. It is thought that these “deregulated” forms escape a critical mechanism that is responsible for the tight regulation of the wild-type protein (Pendergast et al., 1991; Mayer and Baltimore, 1994; reviewed in Van Etten, 1999). Many researchers have suggested that these mutations enable c-Abl to escape regulation by a cellular inhibitor and several lines of evidence have contributed to this hypothesis. First, c-Abl and deregulated forms display equal levels of activity after precipitation or partial purification (Pendergast et al., 1991; Mayer and Baltimore, 1994; Dorey et al., 1999). Moreover, very high levels of expression in cells seem to exhaust regulation, as if through titration of a cellular inhibitor (Pendergast et al., 1991). Expression in a heterologous system, such as in the yeast Schizosaccharomyces pombe, showed no difference in activity between c-Abl and deregulated forms, suggesting the absence of a vertebrate c-Abl inhibitor in fingi (Walkenhorst et al., 1996) and thus supporting this hypothesis. Other elements, such as the identification of a considerable number of proteins binding to the SH3 domain of Abl, have all contributed to the wide acceptance of a cellular inhibitor theory of c-Abl regulation (reviewed in Van Etten, 1999; Brasher and Van Etten, 2000).

There have been some suggestions that Abl might be regulated by intramolecular interactions. The crystal structure of regulated c-Src (reviewed in Sicheri and Kuriyan, 1997) prompted a mutational analysis that supported the possibility of c-Abl being regulated by intramolecular interactions. It was suggested that these intramolecular interactions would be similar to those identified in Src tyrosine kinases and would involve binding of the SH3 domain to the linker between the SH2 and catalytic domains, and the catalytic domain itself (Barilá and Superti-Furga, 1998). However, researchers could not reconcile this theory that c-Abl could be autoinhibited with data showing that c-Abl and deregulated forms of c-Abl had similar activity in vitro. The presence of a cellular inhibitor in the process of c-Abl regulation could not therefore be excluded and remains the dominant view. Moreover, the comparison to the regulation of Src family members lacked explanations for the absence of a regulatory C-terminal tail in c-Abl (Barilá and Superti-Furga, 1998; Brasher and Van Etten, 2000; Van Etten, 1999).

In summary, despite having been the subject of extensive research for more than 20 years, the molecular mechanism by which c-Abl is regulated and its role in the cell remain unknown. Protein tyrosine kinases such as Abl play an essential role in the regulation of normal cellular proliferation and differentiation in multicellular organisms, as evidenced by the common incidence of mutations in genes encoding tyrosine kinase proteins in certain cancers. Given the importance of tyrosine kinase proteins in mammalian diseases and particularly in cancer, there is an urgent need for a better understanding of the way in which medically important proteins, such as c-Abl, and the genes encoding them are regulated. An understanding of the way in which tyrosine kinase proteins are regulated will lead to an understanding of how they become deregulated in disease, enabling effective inhibitors of deregulated forms of protein tyrosine kinases to be developed for the treatment of disease, in particular cancer.

### SUMMARY OF THE INVENTION

The inventors have established that the tyrosine kinase activity of c-Abl is regulated via autoinhibition. Contrary to the teaching in the prior art, regulation of the tyrosine kinase activity of c-Abl does not require an SH3 domain-dependent cellular inhibitor. A region of c-Abl found at the N-terminus of the protein, herein described as a “cap region”, binds intramolecularly to c-Abl and is required to achieve and maintain inhibition of tyrosine kinase activity. This cap region has been found to be absent in all oncogenic forms of Abl and in particular in BCR-Abl resulting in deregulation and increased tyrosine kinase activity. However, it has been found that the cap region can reduce the tyrosine kinase activity of BCR-Abl forms and restore regulation, probably by binding directly to the Abl moiety of the oncogenic form.

Accordingly, the invention provides a tyrosine kinase inhibitor protein consisting of the cap region of a c-Abl protein, or a functional equivalent thereof. The tyrosine kinase inhibitor proteins of the invention are useful candidate agents for the development of drugs to treat disease caused by deregulated tyrosine kinase activity, as well as valuable tools for research into the normal roles of tyrosine kinases in cells.

The cap region has been shown to contain two conserved domains either or both of which appear to be required for it to achieve and maintain inhibition of the tyrosine kinase activity of the c-Abl protein. The first region, referred to herein as the “cap 1 domain”, is at least four amino acids long and appears to be critical for binding the catalytic domain. The second region, herein referred to as the “cap 6 domain”, is also at least four amino acids long and appears to interact with the SH2 and/or SH3 domains.

By a “cap region” of a c-Abl protein is meant a protein sequence of between 20 and 250 amino acids derived from the N-terminal region of a c-Abl protein comprising a cap 1 domain and/or a cap 6 domain or a functional equivalent thereof.

Where both the cap 1 domain and the cap 6 domain are present, they should be appropriately spaced so as to enable the cap 1 domain to bind the catalytic domain of the target tyrosine kinase and the cap 6 domain to bind the SH2 and/or SH3 domains of the target tyrosine kinase. The number of amino acids separating the cap 1 domain and the cap 6 domain may vary provided that the cap 1 and cap 6 domains are spaced appropriately to enable them to bind to the target tyrosine kinase. For example, the cap 1 and cap 6 domains may be separated only by a sufficient number of amino acids necessary to stretch between the catalytic domain and the SH2 and/or SH3 domains. Alternatively, the cap 1 and cap 6 domains may be separated by a larger number of amino acids with the intervening amino acids “looping out” between the binding site of the cap 1 domain and the binding site of the cap 6 domain.

Preferably, the cap 1 domain comprises the sequence KXXG, where X is L or V. Preferably, the cap 6 domain comprises the sequence KENL.

Preferably, the cap region comprises additional conserved regions outside the cap 1 and cap 6 domains. Preferably, the cap region comprises a conserved stretch of amino acids upstream of the cap 6 domain. Preferably, this conserved stretch of amino acids is approximately 9 amino acids long and together with the cap 6 domain forms an alpha helix. Preferably, said conserved stretch of amino acids upstream of the cap 6 domain comprises the amino acid sequence LXEAZRWNS, where X is S or N and Z is A or R.

Preferably, the cap region consists of the N-terminal region of a c-Abl protein and is encoded by the first exon and the first part of the second exon of this gene. More precisely, the cap region preferably consists of the N-terminal region of a c-Abl protein encoded by the first exon and the first 60 to 70 amino acids of the second exon. The cap 1 domain is generally located in the first exon and the cap 6 domain is generally located in the second exon of the Abl gene.

In an even more preferred embodiment, the cap region may be derived from the N-terminal region of any c-Abl protein from any species including, but not limited to, vertebrates and invertebrates. Preferably, the cap region is derived from a mammalian c-Abl protein, preferably a human c-Abl protein or a murine c-Abl protein. The c-Abl protein exists in humans and in mice in two isoforms produced by splicing one of two alternative first exons, type 1a and 1b, to a common second exon. The two isoforms of c-Abl are referred to herein as Abl 1a and Abl 1b in humans and Abl I and Abl IV in mice.

Where the cap region is derived from a human c-Abl protein, it may be derived from either human c-Abl 1a or human c-Abl 1b. Where the cap region is derived from human c-Abl 1a, it preferably consists of amino acid residues 1-61 of the sequence of c-Abl 1a given in FIG. 8 or is a functional equivalent thereof. Where the cap region is derived from human c-Abl 1b, the cap region preferably consists of amino acid residues 1-80 of the sequence of c-Abl 1a given in FIG. 8 or is a functional equivalent thereof.

Where the cap region is derived from a murine c-Abl protein, it may be derived from either murine c-Abl I or murine c-Abl IV. Where the cap region is derived from murine c-Abl I, it preferably consists of amino acids 1-63 of the sequence of c-Abl I as exemplified in FIG. 8 or is a functional equivalent thereof. Where the cap region is derived from murine c-Abl IV, it preferably consists of amino acids 1-80 of c-Abl IV as exemplified in FIG. 8 or is a functional equivalent thereof.

The precise boundaries for the cap region given above are, of course, approximate, in that proteins that have been truncated within or extended beyond these boundaries may still be effective as tyrosine kinase inhibitor proteins, providing that the cap 1 and cap 6 domains have not been deleted. Furthermore, as and when new c-Abl proteins are identified and characterised, the cap regions from these proteins may be used as tyrosine kinase inhibitor proteins according to the invention.

The cap region of c-Abl has been shown to auto-inhibit c-Abl during normal regulation without any requirement for a cellular inhibitor and to restore regulation of tyrosine kinase activity to oncogenic forms of Abl lacking the cap region. Although the Applicant does not wish to be bound by this theory, it is believed that the cap region may also inhibit the tyrosine kinase activity of proteins other than c-Abl. The tyrosine kinase inhibitor proteins of the invention may therefore inhibit any tyrosine kinase protein containing SH2 and SH3 domains. Preferably, the tyrosine kinase inhibitor proteins and functional equivalents of the invention inhibit Abl proteins, preferably oncogenic forms of Abl, including, for example, BCR-Abl. Other tyrosine kinase proteins which may be inhibited by the tyrosine kinase inhibitor proteins and functional equivalents of the invention include members of the src family, such as src, fyn, yes, lyn, lck, blk, hck, fgr and yrk. Other examples of tyrosine kinase proteins which may be inhibited by the tyrosine kinase inhibitor proteins and functional equivalents of the invention include, frk, brk, srm, sad, btk, itk, tec, mkk2, txk, csk, ctk, zap70, syk, fes/fps and fer. Preferably, the oncogenic forms of these proteins are inhibited by the tyrosine kinase inhibitor proteins of the invention or functional equivalents thereof. Examples of oncogenic forms of tyrosine kinase proteins which may be inhibited by the tyrosine kinase inhibitor proteins and functional equivalents of the invention include v-Src and v-Fyn.

Functional equivalents of the tyrosine kinase inhibitor proteins described above are also included within the scope of the invention. The term “functional equivalent” is used herein to describe variants, derivatives and homologues of the tyrosine kinase inhibitor proteins of the invention that retain the ability to inhibit tyrosine kinase proteins.

Functional equivalents of the tyrosine kinase inihibitor proteins of the invention thus include natural biological variants (e.g. allelic variants or geographical variants within the species from which the tyrosine kinase inhibitor proteins are derived). Functionally-equivalent variants of the tyrosine kinase inhibitor proteins of the invention also include, for example, mutants containing amino acid substitutions, insertions or deletions from the wild type protein sequences presented herein. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Be; among Ser and Thr, among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions.

Functionally-equivalent variants with improved function from that of the wild type sequences may also be designed through the systematic or directed mutation of specific residues in the protein sequence. Improvements in function that may be desired will include improvements such as a greater ability to inhibit tyrosine kinase activity.

The term “functional equivalent” also refers to molecules that are structurally similar to the tyrosine kinase inhibitor proteins of the invention or that contain similar or identical tertiary structure. Such functional equivalents may be derived from the natural tyrosine kinase inhibitor proteins or they may be prepared synthetically or using techniques of genetic engineering. In particular, synthetic molecules that are designed to mimic the tertiary structure or active site of the naturally-occurring tyrosine kinase inhibitor proteins of the invention are considered to be functional equivalents. In particular, synthetic molecules which are designed to mimic the tertiary structure of the cap 1 domain and the cap 6 domain in naturally-occurring cap regions are considered to be functional equivalents. Where it is desired that both the cap 1 domain and the cap 6 domain be present, the skilled person will readily be able to design molecular mimetics of cap regions from natural c-Abl proteins which contain the cap 1 and cap 6 domains in the correct spatial conformation to bind and inhibit a target tyrosine kinase.

The term “functional equivalent” also includes derivatives of the tyrosine kinase inhibitor proteins of the invention. By the term “derivative” is meant tyrosine kinase inhibitor proteins that have been modified, for example, by the covalent attachment of non-protein groups. In particular, the term “derivative” includes tyrosine kinase inhibitor proteins of the invention which have undergone post-translation modification. Post-translation modification of c-Abl appears to play a role in controlling its activity (Jackson & Baltimore, 1989). It is therefore postulated that the function of the tyrosine kinase inhibitor proteins of the invention may be affected by post-translation modification and in particular, the covalent attachment of a fatty acly group or a prenyl group. Preferably, the post-translation modification involves covalent attachment of a fatty acyl group or a prenyl group to the tyrosine kinase inhibitor protein. Preferably, derivatives of the tyrosine kinase inhibitor proteins of the invention include myristoylated and palmitoylated derivatives.

The term “functional equivalent” also refers to homologues of the tyrosine kinase inhibitor proteins of the invention. By “homologue” is meant a protein exhibiting a high degree of similarity or identity to the amino acid sequence of the tyrosine kinase inhibitor proteins of the invention. By “similarity” is meant that, at any particular position in the aligned sequences, the amino acid residue is of a similar type between the sequences. By “identity” is meant that at any particular position in the aligned sequences, the amino acid residue is identical between the sequences.

Preferably, homologues possess greater than 40% identity with the naturally-occurring tyrosine kinase inhibitor protein sequences. More preferably, homologues according to the invention show greater than 50%, 60%, 70%, 80%, 90%, 95%, 97%, 98% or 99% sequence identity with the sequence of the natural protein. Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty-11 and gap extension penalty=1]. Tools such as PROSITE (http://expasy.hcuge.ch/sprot/prosite.html), PRINTS http://iupab.leeds.ac.uklbmb5dp/prints.html), Profiles (http://ulrec3.unil.ch/software/PFSCAN_form.html), Pfam (http://www.sanger.ac.uk/software/pfam), Identify (http://dna.stanford.edu/identify/) and Blocks (http://www.blocks.fhcrc.org) databases may also be used to identify homologues, as well as hidden Markov models (HMMs; preferably profile HMMs). Such homologues may include proteins in which one or more of the amino acid residues are substituted with another amino acid residue provided that the function of the protein is retained as a tyrosine kinase inhibitor. Any such substituted amino acid residue may or may not be a naturally occurring amino acid.

In particular, functional equivalents of the tyrosine kinase inhibitor proteins of the invention include cap regions derived from the human paralog of c-Abl, Arg. Preferably, the cap regions derived from Arg consist of the amino acid sequences for the cap regions of Arg 1a and Arg 1b given in FIG. 8.

The term “functional equivalents” also includes active fragments of the naturally-occurring tyrosine kinase inhibitor proteins, variants, derivatives and homologues described above. By “active fragment” is meant any fragment of the tyrosine kinase inhibitor proteins, variants, derivatives or homologues that retain the ability to act as tyrosine kinase inhibitors. Such fragments may include both a cap 1 domain and a cap 6 domain or may include only a cap 1 domain or only a cap 6 domain.

According to a further aspect of the invention, there is provided a fusion protein comprising a tyrosine kinase inhibitor protein of the invention or functional equivalent thereof, as described in any one of the embodiments recited above. The tyrosine kinase inhibitor protein or functional equivalent may be genetically or chemically fused to one or more peptides or polypeptides. Preferably, the tyrosine kinase inhibitor protein or functional equivalent is fused to a marker domain. Preferably, the marker domain is a fluorescent tag, an epitope tag that allows purification by affinity binding, an enzyme tag that allows histochemical or fluorescent labelling, or a radiochemical tag. In one embodiment, the fluorescent tag is a green fluorescent protein (GFP) or a fluorescent derivative thereof such as YFP or CFP (see Prasher et al, (1995), Trends in Genetics, 11(8), 320).

Such fusion proteins will be useful in a variety of methods for establishing the role of the tyrosine kinase inhibitor proteins of the invention. For example, they can be used to facilitate the detection of the tyrosine kinase inhibitor proteins of the invention.

Methods for the generation of fusion proteins are standard in the art and will be known to the skilled reader. For example, most general molecular biology, microbiology recombinant DNA technology and immunological techniques can be found in Sambrook et al., (Molecular Cloning, A Laboratory Manual, Cold Harbor-Laboratory Press, Cold Spring Harbor, N.Y., 2000) or Ausubel et al., (Current Protocols in Molecular Biology, Wiley Interscience, New York, 1991).

Generally, fusion proteins may be most conveniently generated recombinantly from nucleic acid molecules in which two nucleic acid sequences are fused together in frame. These fusion proteins will be encoded by nucleic acid molecules that contain the relevant coding sequence of the fusion protein in question.

The tyrosine kinase inhibitor proteins, functional equivalents and fusion proteins of the invention may be prepared in recombinant form by expression in a host cell. Suitable expression methods are well known to those of skill in the art and many are described in detail by Sambrook J. et al Molecular cloning: a laboratory manual New York: Cold Spring Harbour Laboratory Press, 2000) and Fernandez J. M. & Hoeffler J. P. (Gene expression systems. Using nature for the art of expression ed. Academic Press, San Diego, London, Boston, New York, Sydney, Tokyo, Toronto, 1998.) The proteins and functional equivalents of the present invention can also be prepared using conventional techniques of protein chemistry, for example by chemical synthesis.

According to a further embodiment, the invention provides antibodies that bind to a tyrosine kinase inhibitor protein or functional equivalent, as described above. The invention further provides antibodies that bind to a complex of a tyrosine kinase inhibitor protein and a deregulated tyrosine kinase protein. Antisera and monoclonal antibodies can be made by standard protocols using the tyrosine kinase inhibitor protein or functional equivalent as an immunogen (see, for example, Antibodies: A Laboratory Manual ed. By Harlow and Lane, Cold Spring Harbor Press, 1988). As used herein, the term “antibody” includes fragments of antibodies that also bind specifically to a tyrosine kinase inhibitor protein or functional equivalent thereof. The term “antibody” further includes chimeric and humanised antibody molecules having specificity for the tyrosine kinase inhibitor proteins of the invention and for functional equivalents thereof. Antibodies that bind to tyrosine kinase inhibitor proteins are useful in a variety of methods for elucidating the function of tyrosine kinase proteins and in particular deregulated tyrosine kinase proteins. For example, they can be used to demonstrate the presence of a tyrosine kinase inhibitor protein bound to a tyrosine kinase protein. They can also be used to measure the quantity of a tyrosine kinase inhibitor protein in a cell extract. In some cases, it will be desirable to attach a label group to the antibody in order to facilitate detection. Preferably, the label is an enzyme, a radiolabel or a fluorescent tag. These antibodies may also have medical applications. In particular, an antibody that binds to a complex of a tyrosine kinase inhibitor protein bound to a deregulated tyrosine kinase may act to keep the complex associated, ensuring that the normal tyrosine kinase activity of the protein is restored, reducing its oncogenic capabilities.

The invention also provides a nucleic acid molecule encoding a tyrosine kinase inhibitor protein, functional equivalent thereof, or fusion protein as described above. Specific examples of nucleic acid molecules encoding tyrosine kinase inhibitor proteins according to the invention include those nucleic acid sequences presented in FIG. 9 herein. However, included in this aspect of the invention are nucleic acid molecules that are at least 30%, 40%, 50%, 60% or 70% identical, more preferably, at least 80%, 90%, 95%, 98% or 99% identical over their entire length to a nucleic acid molecule encoding a tyrosine kinase inhibitor protein according to any one of the embodiments of the invention described above. Such nucleic acid molecules include single- or double-stranded DNA, cDNA and RNA, as well as synthetic nucleic acid species. Preferably, the nucleic acid molecules are DNA or cDNA molecules. According to one embodiment, the invention provides a nucleic acid molecule including a nucleic acid sequence presented in FIG. 9.

The nucleic acid sequences will have medical applications. For example, a nucleic acid sequence encoding a tyrosine kinase protein of the invention may be supplied to a patient instead of supplying the tyrosine kinase inhibitor protein directly to the patient. In addition, it has been shown that regulation of a deregulated tyrosine kinase protein lacking the cap region, BCR-Abl, can be restored by integrating a nucleic acid sequence encoding the cap region protein into the gene encoding the tyrosine kinase protein. These results demonstrate that nucleic acid molecules encoding the tyrosine kinase inhibitor proteins of the invention have potential applications in gene therapy.

The invention also includes cloning and expression vectors incorporating the nucleic acid molecules. Such expression vectors may additionally incorporate regulatory sequences such as enhancers, promoters, ribosome binding sites and termination signals in the 5′ and 3′ untranslated regions of genes, that are required to ensure that the coding sequence is properly transcribed and translated, or to regulate the expression of the protein relative to the growth of the cell in which it is expressed. Also, control sequences may be included that encode signal peptides or leader sequences. These leader or control sequences may be removed by the host during post-translational processing. In some cases, the vectors may incorporate signal sequences which direct the expressed proteins to specific subcellular locations. For example, it may be desirable to assess the effect of a tyrosine kinase inhibitor protein in the nucleus, in which case a nuclear localisation signal may be included in the vector.

Vectors according to the invention include plasmids and viruses (including both bacteriophage and eukaryotic viruses), as well as other linear or circular DNA carriers, such as those employing transposable elements or homologous recombination technology. Many such vectors and expression systems are known and documented in the art (see, for example, Fernandez J. M. & Hoeffler J. P. in Gene expression systems. Using nature for the art of expression ed. Academic Press, San Diego, London, Boston, New York, Sydney, Tokyo, Toronto, 1998). Suitable viral vectors include baculovirus-, adenovirus- and vaccinia virus-based vectors.

Suitable hosts for recombinant expression include commonly used prokaryotic species, such as E. coli, or eukaryotic yeasts that can be made to express high levels of recombinant proteins and that can easily be grown in large quantities. Mammalian cell lines grown in vitro are also suitable, particularly when using virus-derived expression systems. Another suitable expression system is the baculovirus expression system that involves the use of insect cells as hosts. An expression system may also constitute host cells that have the appropriate encoding nucleic acid molecules incorporated into their genome. Proteins may also be expressed in vivo, for example, in insect larvae or in mammalian tissues.

A variety of techniques may be used to introduce the vectors according to the present invention into prokaryotic or eukaryotic host cells. Suitable transformation or transfection techniques are well described in the literature (see, for example, Sambrook et al, Molecular cloning: a laboratory manual New York: Cold Spring Harbour Laboratory Press, 2000; Ausubel et al, Current Protocols in Molecular Biology, Wiley Interscience, New York, 1991; Spector, Goldman & Leinwald, Spector et al Cells, a laboratory manual; Cold Spring Harbour Laboratory Press, 1998). In eukaryotic cells, expression systems may either be transient (e.g. episomal) or permanent (such as by chromosomal integration) according to the needs of the system.

The invention also provides antisense nucleic acid molecules which hybridise under high stringency hybridisation conditions to the nucleic acid molecules encoding the tyrosine kinase inhibitor proteins or functional equivalents thereof. High stringency hybridisation conditions are defined herein as overnight incubation at 42° C. in a solution comprising 50% formamide, 5×SSC (150 mM NaCl, 15 mM trisodium citrate), 50 mM sodium phosphate (pH7.6), 5× Denhardts solution, 10% dextran sulphate, and 20 microgram/ml denatured, sheared salmon sperm DNA, followed by washing the filters in 0.1×SSC at approximately 65° C.

In a preferred embodiment of this aspect of the invention, a label capable of being detected is attached to these antisense nucleic acid molecules. Preferably, the label is selected from the group consisting of radioisotopes, fluorescent compounds and enzymes.

These antisense nucleic acid molecules may be used as probes to detect defects in the nucleic acid molecules encoding a tyrosine kinase protein to which the tyrosine kinase inhibitor protein of the invention binds. The antisense nucleic acid molecules of the invention may therefore be useful in diagnostic assays. For example, it has been shown that the cap region is generally missing in oncogenic forms of the tyrosine kinase protein Abl, such as BCR-Abl but that reintroduction of a nucleic acid molecule encoding the cap region restores regulation. The antisense nucleic acid molecules of the invention may therefore be useful for detecting whether a cap region is present in the nucleic acid molecule encoding Abl and to screen for mutation in the cap region of an Abl protein. The antisense nucleic acid molecules can also be used to detect whether a nucleic acid encoding a cap region has been successfully introduced into a gene encoding an oncogenic form of Abl, such as BCR-Abl.

The invention also includes transformed or transfected prokaryotic or eukaryotic host cells containing a nucleic acid molecule encoding a tyrosine kinase inhibitor protein or functional equivalent thereof as described above or an antisense nucleic acid molecule which hybridises to such a nucleic acid molecule.

A further aspect of the invention provides a method for preparing a tyrosine kinase inhibitor protein or a functional equivalent thereof or a fusion protein as defined above, which comprises culturing a host cell containing a nucleic acid molecule encoding a tyrosine kinase inhibitor protein or functional equivalent thereof or a fusion protein according to the invention under conditions whereby said protein is expressed and recovering said protein thus produced.

As the skilled reader will appreciate, the identification of the tyrosine kinase inhibitor proteins of the present invention and, in particular, the identification of the role of such proteins in the autoinhibition of c-Abl will result in an increased understanding of the way in which tyrosine kinase proteins become deregulated in diseases such as cancer. Although the applicant does not wish to be bound by any specific theory, it is considered possible that the normal autoinhibition of c-Abl by its N-terminal cap region may be disrupted by molecules that prevent the cap region from interacting with the catalytic and SH2 and/or SH3 domains. Such activator compounds will increase the tyrosine kinase activity of c-Abl and may be useful targets for the development of drugs. Once such activator compounds have been identified, it may be possible to identify modulator compounds that inhibit the ability of the activator compounds to disrupt normal autoinhibition of c-Abl by its N-terminal cap region.

A further aspect of the invention therefore provides a method of identifying an activator compound that inhibits autoinhibition of c-Abl by the cap region comprising contacting c-Abl with a candidate activator compound and assessing whether binding between the cap region of c-Abl and the catalytic and SH2 and/or SH3 domains of c-Abl has been inhibited.

There are numerous methods of determining if the candidate activator compounds binds at a site on the c-Abl protein that prevents binding between the cap region and the catalytic domain and the SH2 and/or SH3 domains. In particular, competition assays can be used to determine if a candidate activator compound prevents autoinihibition by the cap region.

In order to identify candidate activator compounds of the present invention, both the cap region of c-Abl and c-Abl proteins lacking the cap region can be used to screen libraries of compounds in any of a variety of drug screening techniques.

Candidate activator compounds may be isolated from, for example, cells, cell-free preparations, chemical libraries, or natural product mixtures. Suitable compounds might include polypeptides, such as enzymes, receptors, antibodies, and structural or functional mimetics of these polypeptides, including peptides and peptidomimetics. These may either be natural compounds, isolated from natural sources, or may be synthetic or recombinant.

The c-Abl cap region that is employed in such a screening technique may be free in solution, affixed to a solid support, borne on a cell surface, or located intracellularly. The adherence of a candidate activator compound to a surface bearing the c-Abl cap region can be detected by means of a label directly or indirectly associated with the candidate activator compound or in an assay involving competition with a labelled competitor. In general, such screening procedures may involve using appropriate cells or cell membranes that express the appropriate protein, that are then contacted with a candidate activator protein to observe binding. Binding may be detected, for example, using a yeast 2-hybrid screen.

Another technique for drug screening which may be used provides for high throughput screening of compounds having suitable binding affinity to the protein of interest (for example, see International patent application WO84/03564). In this method, large numbers of different small test compounds are synthesised on a solid substrate, which may then be reacted with the c-Abl cap region and washed. One way of immobilising the target protein is to use non-neutralising antibodies. Bound protein may then be detected using methods that are well known in the art, including using biophysical techniques such as surface plasmon resonance and spectroscopy. Purified protein can also be coated directly onto plates for use in the aforementioned drug screening techniques.

In a preferred embodiment, a competition assay is performed wherein the c-Abl cap region or a c-Abl protein lacking the cap region is attached to a solid support. Any suitable solid support may be used, such as, for example, sepharose beads. Methods for linking proteins to solid supports are well known to those skilled in the art.

For example, when the c-Abl cap region is attached to a solid support, the detection of binding of a c-Abl protein lacking the cap region can easily be achieved, for example using a labelled antibody specific for the SH2 domain or the SH3 domain. Alternatively, a c-Abl protein lacking a cap region may be attached to the solid support and detection of the binding of the cap region can be achieved using a labelled antibody specific for the cap region. Suitable labels include: enzymes, such as horseradish peroxidase (HRP) and chloramphenicol acetyl transferase (CAT); digoxygenin (DIG); fluorescein; and radioisotopes such as ¹²⁵I, ³H and ¹⁴C.

Once a candidate activator compound has been identified using the assay of the present invention, it will be desirable to test the activator compound in a cell containing c-Abl in order to determine its effect on the tyrosine kinase activity of c-Abl.

The invention further provides a method for identifying a modulator compound that restores autoinhibition of c-Abl by the cap region comprising contacting c-Abl and an activator compound, as described above, with a candidate modulator compound and assessing whether binding between the cap region of c-Abl and the catalytic and SH2 and/or SH3 domains of c-Abl is restored.

Candidate modulator compounds may be identified by conducting screening assays to identify compounds that interact with the activator compounds. The ability of a modulator compound to compete with an activator compound can then be assessed using any method known to the person skilled in the art. In particular, competitive assays such as those described above may be carried out in the presence of an activator compound and then in the presence of an activator compound and a modulator compound. Preferably, the step of assessing whether binding between the cap region and the catalytic domain and the SH2 and/or SH3 domains of c-Abl has been restored comprises detecting the restoration of normal (low) tyrosine kinase activity.

The invention further provides a compound that is an activator compound or a modulator compound, identified or identifiable by the screening method mentioned above. It will be understood that modulators of the invention are not limited to those identified by the above method but include any activator compounds which activate c-Abl by inhibiting binding between the cap region of c-Abl and the catalytic and SH2 and/or SH3 domains, as well as any modulator compounds that restore binding by inhibiting an activator compound.

The identification of the function of the cap region of c-Abl protein as an inhibitor of tyrosine kinase activity allows tyrosine kinase proteins to be regulated in vivo, for example, as some form of therapy, or in vitro, for example, to modulate the activity of protein tyrosine activity in tissue culture. According to one aspect of this embodiment of the invention, there is thus provided a method of modulating the activity of a tyrosine kinase protein comprising providing a cell with a tyrosine kinase inhibitor protein or a functional equivalent thereof, a nucleic acid molecule encoding a tyrosine kinase inhibitor protein, an antisense nucleic acid molecule that binds to a nucleic acid molecule encoding a tyrosine kinase protein, an activator compound or a modulator compound as described above. Preferably, the target protein tyrosine kinase is an oncogenic tyrosine kinase protein, preferably an oncogenic form of Abl, preferably BCR-Abl.

According to a further aspect of the invention, there is provided the use of the cap region of a c-Abl protein, as defined above, as a tyrosine kinase inhibitor.

The ability to modulate tyrosine kinase activity in cells and in particular to decrease the tyrosine kinase activity of oncogenic tyrosine kinase proteins will be a useful tool for researchers seeking to understand how deregulation of tyrosine kinase proteins results in diseases such as cancer.

As referred to above, the tyrosine kinase inhibitor proteins, functional equivalents and modulators of the invention have a wide variety of potential medical applications. In particular, the tyrosine kinase inhibitor proteins of the invention have applications in the treatment of diseases associated with deregulated tyrosine kinase activity since they are able to restore regulated tyrosine kinase activity.

Nucleic acids encoding the tyrosine kinase inhibitor proteins of the invention or functional equivalents thereof may also be introduced to treat such diseases. The nucleic acid molecules may be used to express the tyrosine kinase inhibitor protein in a target cell. Alternatively, a specific embodiment of the invention involves integrating the nucleic acid encoding the tyrosine linase inhibitor protein into a gene encoding a deregulated tyrosine kinase protein which has no cap region. Preferably, the tyrosine kinase may be a deregulated form of c-Abl, preferably BCR-ABL.

Accordingly, the invention further provides a tyrosine kinase inhibitor protein or a functional equivalent thereof, a nucleic acid molecule encoding said tyrosine kinase inhibitor protein or functional equivalent, an antisense nucleic acid molecule, an activator compound or a modulator compound as described above, for use as a pharmaceutical.

A further aspect of the invention includes a pharmaceutical composition comprising a tyrosine kinase inhibitor protein or a functional equivalent thereof, a nucleic acid molecule encoding said tyrosine kinase inhibitor protein or functional equivalent, an antisense nucleic acid molecule, an activator compound or a modulator compound according to any one of the embodiments of the invention recited above, in conjunction with a pharmaceutically-acceptable carrier molecule.

Carrier molecules may be genes, polypeptides, antibodies, liposomes, polysaccharides, polylactic acids, polyglycolic acids and inactive virus particles or indeed any other agent provided that the carrier does not itself induce toxicity effects or cause the production of antibodies that are harmful to the individual receiving the pharmaceutical composition. Carriers may also include pharmaceutically acceptable salts such as mineral acid salts (for example, hydrochlorides, hydrobromides, phosphates, sulphates) or the salts of organic acids (for example, acetates, propionates, malonates, benzoates). Pharmaceutically acceptable carriers may additionally contain liquids such as water, saline, glycerol, ethanol or auxiliary substances such as wetting or emulsifying agents, pH buffering substances and the like. Carriers may enable the pharmaceutical compositions to be formulated into tablets, pills, dragees, capsules, liquids, gels, syrups, slurries, suspensions to aid intake by the patient. A thorough discussion of pharmaceutically acceptable carriers is available in Remington's Pharmaceutical Sciences (Mack Pub. Co., N.J. 1991).

The amount of the active compound in the composition should also be in a therapeutically-effective amount. The phrase “therapeutically effective amount” used herein refers to the amount of agent needed to treat or ameliorate a targeted disease or condition. An effective initial method to determine a “therapeutically effective amount” may be by carrying out assays in the transgenic organism model, although more accurate tests must be carried out on the target organism if initial tests are successful. The transgenic organism model may also yield relevant information such as the preferred routes of administration that will lead to maximum effectiveness. The exact therapeutically-effective dosage will generally be dependent on the patient's status at the time of administration. Factors that may be taken into consideration when determining dosage include the severity of the disease state in the patient, the general health of the patient, the age, weight, gender, diet, time and frequency of administration, drug combinations, reaction sensitivities and the patient's tolerance or response to the therapy. The precise amount can be determined by routine experimentation but may ultimately lie with the judgement of the clinician. Generally, an effective dose will be from 0.01 mg/kg (mass of drug compared to mass of patient) to 50 mg/kg, preferably 0.05 mg/kg to 10 mg/kg. Compositions may be administered individually to a patient or may be administered in combination with other agents, drugs or hormones.

Uptake of a pharmaceutical composition by a patient may be initiated by a variety of methods including, but not limited to enteral, intra-arterial, intrathecal, intramedullary, intramuscular, intranasal, intraperitoneal, intravaginal, intravenous, intraventricular, oral, rectal (for example, in the form of suppositories), subcutaneous, sublingual, transcutaneous applications (for example, see WO98/20734) or transdermal means. Gene guns or hyposprays may also be used to administer pharmaceutical compositions. Typically, however, the therapeutic compositions may be prepared as injectables, either as liquid solutions or suspensions; solid forms suitable for solution in, or suspension in, liquid vehicles prior to injection may also be prepared. Direct delivery of the compositions can generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

The invention also includes the use of a tyrosine kinase inhibitor protein or a functional equivalent thereof, a nucleic acid molecule encoding said tyrosine kinase inhibitor protein or functional equivalent thereof, an antisense nucleic acid molecule, an activator compound or a modulator compound as described above, in the manufacture of a medicament for treating a disease or a condition associated with aberrant tyrosine kinase activity.

The tyrosine kinase inhibitor proteins and functional equivalents, the nucleic acid molecules encoding these tyrosine kinase inhibitor proteins and functional equivalents, the antisense nucleic acid molecules, the activator compounds and the modulator compounds of the invention will be useful in the manufacture of medicaments for treating diseases associated with aberrant tyrosine kinase activity.

The tyrosine kinase inhibitor proteins and functional equivalents, the nucleic acid molecules encoding these tyrosine kinase inhibitor proteins and functional equivalents, and the modulator compounds of the invention will be useful in the manufacture of medicaments for treating diseases associated with an aberrantly high level of tyrosine kinase activity. Preferably, such a disease is cancer, more preferably leukaemia The antisense nucleic acid molecules and activator compounds of the invention will be useful in the manufacture of medicaments for treating diseases associated with an aberrantly low level of tyrosine kinase activity, such as agammaglobulinemia. The tyrosine kinase inhibitor proteins and functional equivalents, the nucleic acid molecules encoding these tyrosine kinase inhibitor proteins and functional equivalents, the antisense nucleic acid molecules, the activator compounds and the modulator compounds of the invention will also be useful in the treatment of neurological disorders caused by aberrant tyrosine kinase activity.

According to a still further aspect of the invention, there is provided a method of treating a disorder or disease associated with aberrant tyrosine kinase activity in a patient, comprising administering to the patient a tyrosine kinase inhibitor protein or functional equivalent thereof, a nucleic acid molecule encoding said tyrosine kinase inhibitor protein or functional equivalent, an antisense nucleic acid molecule, an activator compound, a modulator compound or a composition as described above in a therapeutically-effective amount. Preferred patients are mammals, more preferably humans. Preferably, the disorder or disease associated with aberrant tyrosine kinase activity is a neurological disorder, agammaglobulinemia or cancer, preferably leukaemia.

The invention further comprises a method of diagnosing a condition associated with an aberrant activity of a tyrosine kinase protein comprising measuring the level of an aberrant tyrosine kinase protein in a cell sample obtained from a patient using a tyrosine kinase inhibitor protein of the invention. As indicated above, the tyrosine kinase inhibitor proteins of the invention are supposed to inhibit the oncogenic forms of tyrosine kinase proteins but not the cellular forms. For example, the N-terminal cap region of c-Abl can inhibit BCR-Abl, probably by binding to it. In contrast, the N-terminal cap region of c-Abl does not bind c-Abl, presumably because the binding site is already occupied by the endogenous c-Abl cap region. Accordingly, the presence of an oncogenic tyrosine kinase may be diagnosed by detecting the presence and the level of complexes comprising a tyrosine kinase inhibitor protein of the invention, bound to an oncogenic tyrosine kinase. Preferably, the level of the complex in the sample is measured using antibodies against the tyrosine kinase inhibitor protein or the complex, as described previously.

The invention further comprises a method of diagnosing a condition associated with an aberrant activity of a tyrosine kinase protein comprising using a nucleic acid molecule or an antisense nucleic acid molecule, as described above, to screen for mutations in the cap region of the protein tyrosine kinase Abl.

In another embodiment of the invention, a nucleic acid molecule as described above may be used to create a transgenic animal, most conunonly a rodent. The modification of the animal's genome may either be done locally, by modification of somatic cells or by germ line therapy to incorporate inheritable modifications. Such transgenic animals may be particularly useful in assessing whether integration of nucleic acid molecules encoding tyrosine kinase inhibitor proteins of the invention can be used to restore regulation of tyrosine kinase in an animal model in which the tyrosine kinase activity has been deregulated, causing cancer. The transgenic animals will also be useful in assessing the effectiveness of modulator compounds, as described above.

As indicated above, the tyrosine kinase inhibitor proteins of the invention will be useful in developing a more detailed understanding of the role of tyrosine kinases and the way in which they are regulated. The tyrosine kinase inhibitor proteins of the invention are the result of the initial discovery by the inventors that the cap region of c-Abl is involved in its auto-inhibition. The inventors have also developed a technique for assessing the effects of deregulating c-Abl in particular. Specifically, they have developed a c-Abl protein that contains a mutation that introduces a Tobacco-Etch-Virus (TEV) protease cleavage site roughly at the boundary between the cap region and the SH3 domain. The mutation does not affect the normal function of c-Abl but treatment of cells expressing this protein with TEV protease results in cleavage of the cap region and deregulation of the tyrosine kinase activity of c-Abl in vitro. This TEV construct hence has clear applications in the study of the effects of the activation of c-Abl activity.

Accordingly, according to a further aspect of the invention, there is provided a c-Abl protein comprising a protease cleavage site located near the boundary of the cap region and the SH3 domain. Preferably, said protease cleavage site is a TEV protease cleavage site. Where the c-Abl protein is a human type 1b c-Abl protein, the cleavage site is preferably located at residues 82-85, such that cleavage occurs at residue 78, cleaving residues 1-77 of the N-terminal domain.

According to a further aspect of the invention, there is provided a fusion protein comprising a c-Abl protein comprising a protease cleavage site bound to a marker domain, as described above in respect of the tyrosine inhibitor proteins in general.

According to a further aspect of the invention, there is provided a method for activating the tyrosine kinase activity of c-Abl in a cell comprising supplying the cell with a c-Abl protein comprising a protease cleavage site as described above and supplying the cell with a protease. Preferably, the method further comprises removing the cleaved N-terminal domain to ensure that the activated c-Abl protein is not inhibited by binding of the cleaved N-terminal domain to it.

There is also provided a method for producing an activated c-Abl protein comprising cleaving a c-Abl protein comprising a protease cleavage site, as described above, with a protease and isolating the cleaved C-terminal region of the c-Abl protein.

There is also provided a method of producing a tyrosine kinase inhibitor protein according to the invention comprising cleaving a c-Abl protein comprising a protease site as described above with a protease and isolating the cleaved N-terminal cap region of the c-Abl protein.

The invention also provides nucleic acid molecules encoding the c-Abl proteins and fusion proteins comprising a protease domain as described above, as well as vectors comprising the nucleic acid molecules.

There is also provided a method for activating a c-Abl protein comprising introducing a nucleic acid molecule encoding a c-Abl protein containing a protease cleavage site into a cell under conditions in which it is expressed and supplying said cell with a protease.

The invention further provides a method for producing an activated c-Abl protein comprising introducing a nucleic acid molecule encoding a c-Abl protein containing a protease cleavage site into a cell under conditions in which it is expressed, supplying the cell with a protease and isolating the cleaved activated C-terminal region of the c-Abl protein.

The invention also provides a method for producing a tyrosine kinase inhibitor protein according to the aspects of the invention described above, comprising introducing a nucleic acid molecule encoding a c-Abl protein containing a protease cleavage site into a cell under conditions in which it is expressed, supplying said cell with a protease and isolating the cleaved N-terminal cap region of the c-Abl protein.

The invention further provides a transgenic animal comprising a nucleic acid encoding a c-Abl protein containing a protease cleavage domain, as described above. These transgenic animals are useful as they will enable researchers to assess the effect of activating the tyrosine kinase activity of c-Abl in vivo. According to a further aspect of the invention, there is provided a method for activating tyrosine kinase activity of c-Abl in vivo comprising supplying a transgenic animal comprising a nucleic acid encoding a c-Abl protein containing a protease cleavage domain, as described above, with a protease. This method can be conducted using any genetic background in order to assess the effect of additional mutations on c-Abl activation. In addition, the transgenic animal in which the tyrosine kinase activity of c-Abl has been activated can be used in in vivo screening assays to identify compounds that restore autoinhibition of c-Abl The invention therefore provides an in vivo method for screening for a compound that restores autoinhibition of c-Abl comprising activating the tyrosine kinase activity of c-Abl in vivo in a transgenic animal as described above, supplying the transgenic animal with a candidate compound and assessing the effect of the candidate compound on the tyrosine kinase activity in the cells of the transgenic animal. The candidate compound may be any suitable compound. Preferably, the screening method of the invention is used to assess the in vivo effectiveness of modulator compounds identified by the methods described previously.

Various aspects and embodiments of the present invention will now be described in more detail by way of example, with particular reference to the inhibition of the tyrosine kinase, Abl. It will appreciated that modification of detail may be made without departing from the scope of the invention.

### BRIEF DESCRIPTION OF THE FIGURES

FIG. 1: Autoinhibition of c-Abl

A. c-Abl and Abl-PP were expressed by in vitro translation in wheat germ extract. Total protein extract (left panel) and anti-Abl immunoprecipitates (right panel) were separated by SDS-PAGE, blotted to nitrocellulose and probed with anti-Abl and anti-phosphotyrosine antibodies.

B. S. pombe strain SP200 was transformed with a vector expressing c-Abl, ΔSH3-Abl or Abl-PP under control of a thiamine repressable promotor. Sixteen (left panel) and twenty-four hours (right panel) after induction of protein expression by thiamine removal, cells were lysed by boiling in SDS-sample buffer. Lysates were separated by SDS-PAGE, blotted to nitrocellulose and probed with anti-Abl and anti-phosphotyrosine antibodies.

C. HEK293 cells were transiently transfected with SV40-driven c-Abl and Abl-PP expression plasmids. Forty hours after transfection cells were lysed and Abl protein was immunopurified using covalently coupled anti-Abl antibodies. Abl and co-purifying proteins were eluted by pH shock, separated by 4-15% gradient SDS-PAGE and visualised by colloidal Coomassie staining (right panel). The identity of Abl proteins was confirmed by mass spectrometry. Purified c-Abl and Abl-PP proteins were assayed for their catalytic activity by in vitro kinase assay using GST-Crk as exogenous substrate. Bands were excised and incorporated radioactivity was measured by scintillation counting. The histograph shows the catalytic activity of purified Abl-PP compared to purified c-Abl (mean with SD of two experiments done in triplicate, left panel). Part of the Abl protein used for the in vitro kinase assay was blotted and probed with anti-Abl and anti-phosphotyrosine antibodies (middle panels). An autoradiograph showing an example of the incorporation of radioactivity in GST-Crk is presented.

FIG. 2. The core element for c-Abl regulation

A. Schematic diagram representing the different generated Abl proteins. The SH3, SH2 and catalytic domains as well as the last exon region are shown as boxes. P242 and P249 Abl-PP and derivatives.

B. HEK293 cells were transiently transfected with the indicated SV40-driven Abl expression constructs. Forty hours after transfection cells were lysed and total protein extract was analysed by anti-Abl and anti-phosphotyrosine immunoblotting.

C. Abl protein was immunoprecipitated from total cell extract using anti-Abl antibodies, blotted to nitrocellulose and probed with anti-Abl and anti-phosphotyrosine antibodies (left panels). Abl immunoprecipitates were assayed for catalytic activity by in vitro kinase assay using GST-Jun as substrate. The histograph shows the catalytic activity (mean with SD of three experiments done in duplicate) of the Abl constructs relative to Abl M1-K531.

FIG. 3. Autoinhibition of c-Abl by its N-terminus

A. Schematic diagram representing the different generated Abl proteins. The SH3, SH2 and catalytic domains as well as the last exon region are shown as boxes. P242 and P249 indicate the two proline residues in the SH2-catalytic domain linker that are mutated in Abl-PP and derivatives.

B. HEK293 cells were transiently transfected with c-Abl or the indicated Abl expression constructs. Forty hours after transfection cells were lysed and the resulting total protein extract was analysed by anti-Abl and anti-phosphotyrosine immunoblotting.

C. Abl protein was immunoprecipitated from total cell extract using anti-Abl antibodies, blotted to nitrocellulose and probed with anti-Abl and anti-phosphotyrosine antibodies (left panels). Abl immunoprecipitates were assayed for catalytic activity by in vitro kinase assay using GST-Crk as substrate. The histograph shows the catalytic activity (mean with SD of three experiments done in duplicate) of the indicated Abl constructs relative to c-Abl.

FIG. 4. In vitro binding of the N-terminal region to Abl protein

A. c-Abl, Abl ΔM1-D81 or Abl-PP ΔM1-D81 were expressed in HEK293 cells. Cell lysates were incubated with the indicated GST fusion proteins bound to glutathion-Sepharose beads. Adsorbates were analysed by SDS-PAGE followed by anti-Abl immunoblotting (left panel). Inputs are shown for Abl (right panel); identical amounts of GST fusion proteins were used for each pull-down as quantified by Coomassie blue staining (data not shown).

B. HA-tagged pieces of Abl were expressed in HEK293 cells. Cell lysates were incubated with the indicated GST fusion proteins as described in A. Adsorbates were analysed by SDS-PAGE followed by anti-HA immunoblotting.

FIG. 5. Mutagenesis analysis of the N-terminal cap

A. Alignment of the c-Abl type 1a and 1b N-terminal regions. Indicated are the residues (groups of four residues in a row) which are mutated to alanine for the different cap mutants (cap 1-6).

B. Abl cap mutant proteins and the indicated Abl constructs were transiently expressed in HEK293 cells. Anti-Abl immunoprecipitates were analysed by anti-Abl and anti-phosphotyrosine immunoblotting (left panels) and assayed for catalytic activity by in vitro kinase assay using GST-Crk as substrate (right panel). The histograph shows the fold of activation of the different constructs compared to c-Abl (mean with SD of two experiments done in duplicate).

C. HA-tagged Abl protein pieces (SH3-SH2-CD and CD) were expressed in HEK293 cells. Cell lysates were incubated with the indicated GST fusion proteins bound to glutathion-Sepharose beads. Adsorbates were analysed by SDS-PAGE followed by anti-HA immunoblotting.

FIG. 6. Role of the N-terminal cap of c-Abl

A. Schematic diagram showing the structure of the N-terminus of c-Abl. The SH3, SH2 and catalytic domains as well as the last exon region are represented by boxes. The amino acid sequence of the N-terminus of the c-Abl type 1b protein is shown. The bold arrow indicates the beginning of the βa strand of the SH3 domain, the engineered TEV cleavage site is marked in black and the arrowhead indicates the exact position of cleavage by TEV protease.

B. Abl-TEV and indicated constructs were expressed in HEK293 cells and immunoprecipitated with anti-Abl antibodies. Immunecomplexes were incubated without (−) or with (+) TEV protease, separated by SDS-PAGE, blotted to nitrocellulose and probed with anti-Abl antibodies (upper panel). Immunecomplexes were assayed for catalytic activity by in vitro kinase assay using GST-Crk as substrate (lower panel). The histograph shows the fold of activation induced by TEV protease (mean with SD of three independent experiments). This was calculated by dividing the catalytic activity of Abl protein after TEV treatment by the catalytic activity without TEV incubation.

C. Model of the 3-dimensional arrangement of c-Abl including the N-terminal regulatory “cap” based on known structures of the SH3 domain, the SH12 domain and the catalytic domain of Abl and on regulated Src. The N-terminal “cap” is represented as a rod structure which may bind to the “north-face” of Abl thus stabilising the regulated structure in which the SH3 domain is bound via the SH2-catalytic domain linker to the catalytic domain. The arrowhead indicates the position of the engineered TEV cleavage site.

FIG. 7. Inhibition of catalytic activity by the N-terminal cap

A. Abl ΔM1-D81 and Abl-PP ΔM1-D81 were transiently expressed in HEK293 cells and immunoprecipitated using anti-Abl antibodies. Immune complexes were incubated for 2 hours with kinase assay buffer (−) or with equal amounts of GST fusion protein (GST or GST Abl 1b+2) dialysed against kinase assay buffer, and subsequently assayed for catalytic activity by in vitro kinase assay using GST-Crk as substrate. The histograph shows the percentage of inhibition (mean with SD of three experiments done in duplicate).

B. HEK293 cells were transfected with the indicated constructs. After an immunoprecipitation using anti-Abl antibodies, the immunecomplexes were analysed by western-blot anti-Abl (upper panel) or anti-p412 (lower panel).

C. Effect of the cap on the catalytic activity of BCR-Abl or ACC BCR-Abl tested by in vitro kinase assay using GST-Crk as a substrate. The activity of BCR-Abl and ACC BCR-Abl is set to 100%, the histograph shows the variation of catalytic activity when the cap is restored in BCR-Abl or in ACC BCR-Abl (mean of 2 independent experiments done in duplicate with SD).

FIG. 8: Alignment of the amino acid sequences of the cap regions for human c-Abl 1a and 1b, human Arg 1a and 1b, and murine c-Abl I and IV. The positions of the cap 1 and cap 6 domains are shown.

FIG. 9: Alignment of the nucleic acid sequences of the cap regions for human c-Abl 1a and 1b, human Arg 1a and 1b, and murine c-Abl I and IV.

### EXAMPLES

1) Results

Expression of c-Abl in Non-Vertebrate Systems

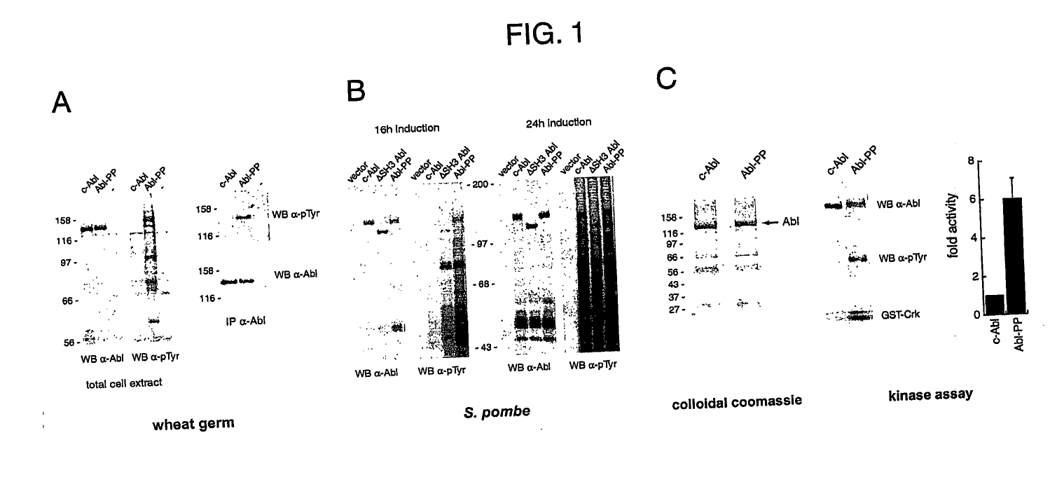

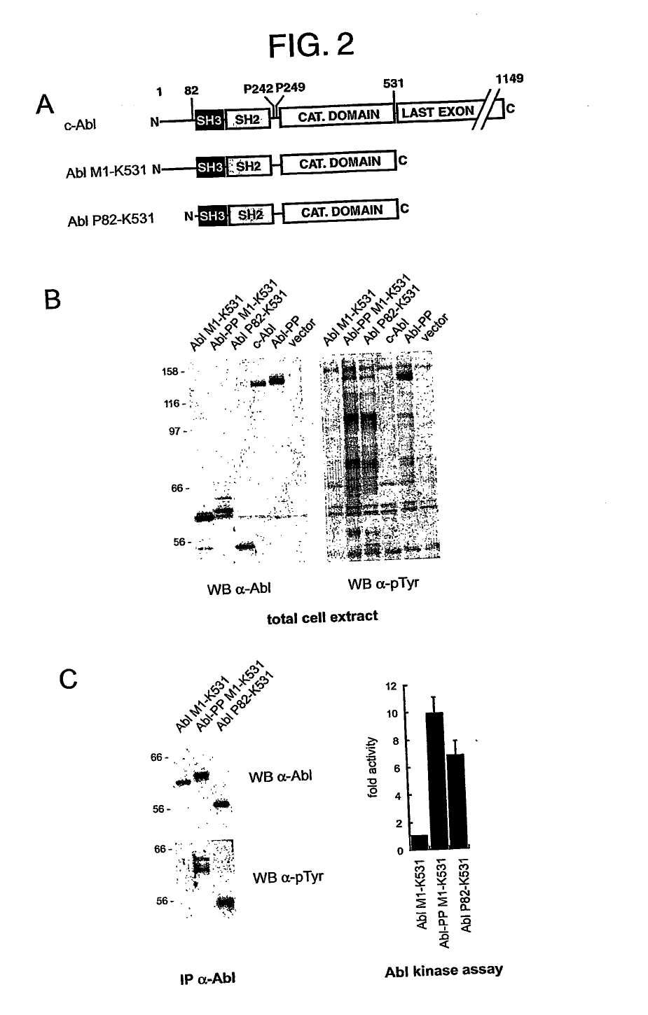

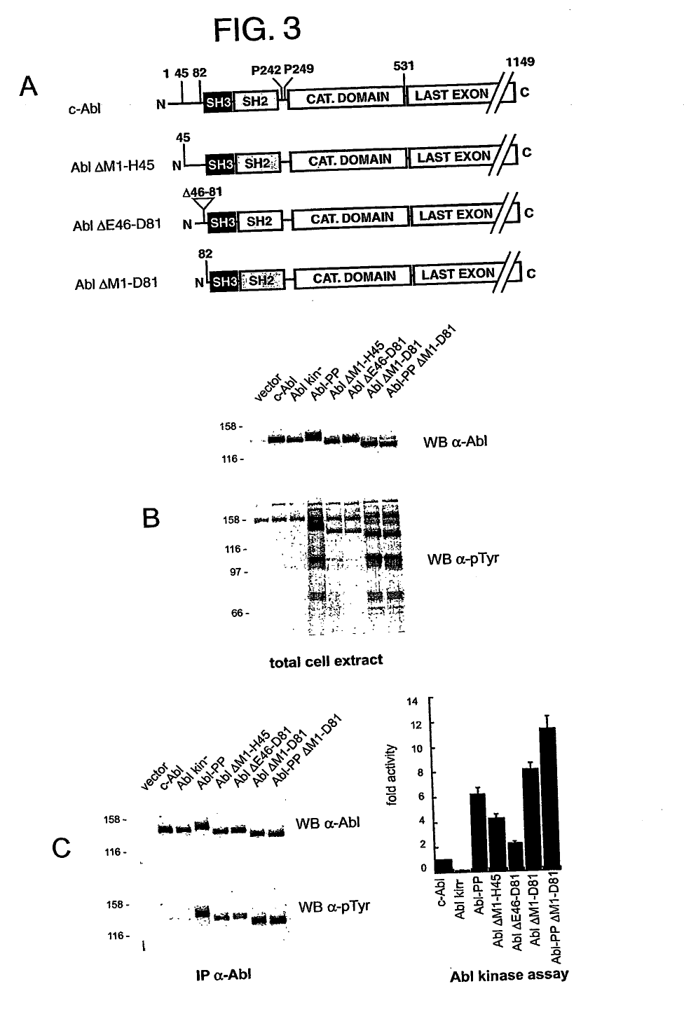

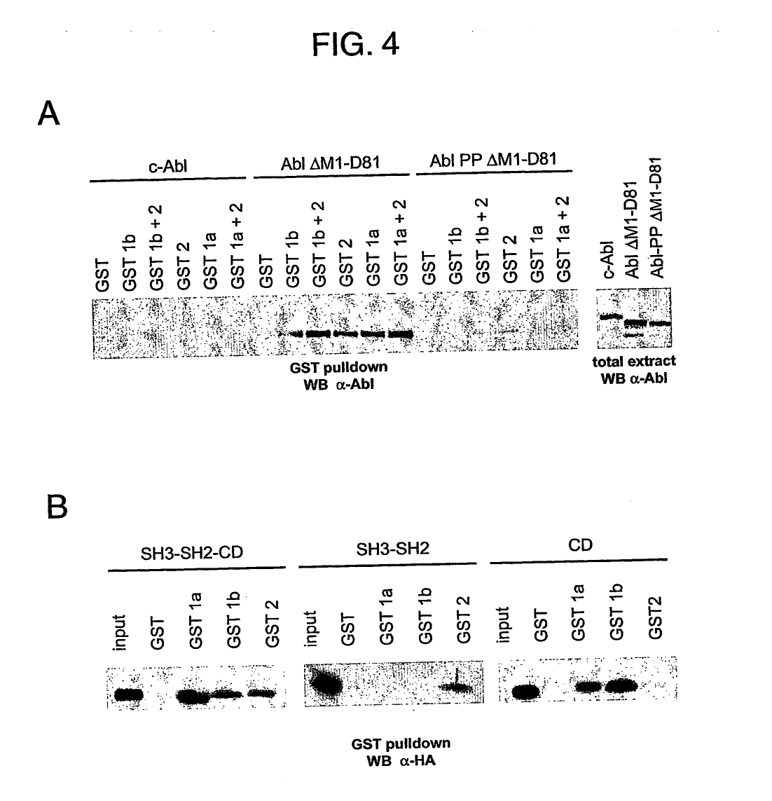

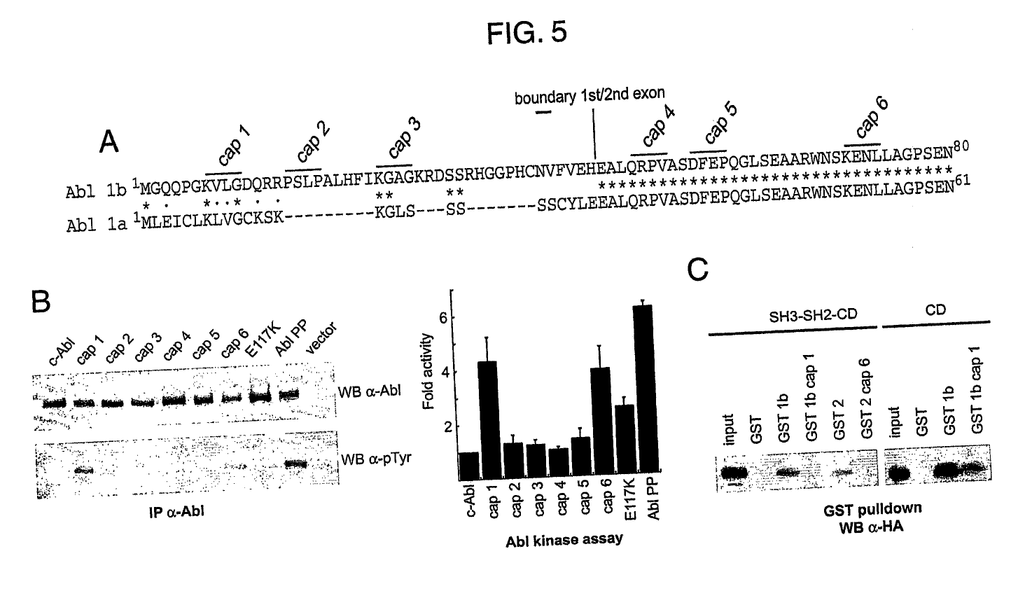

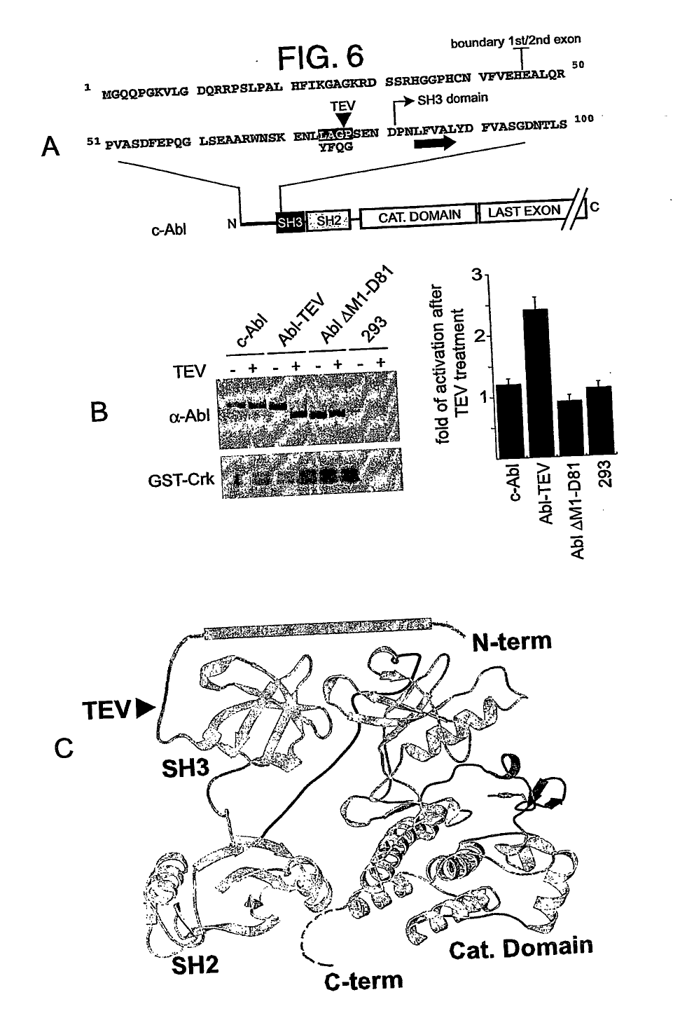

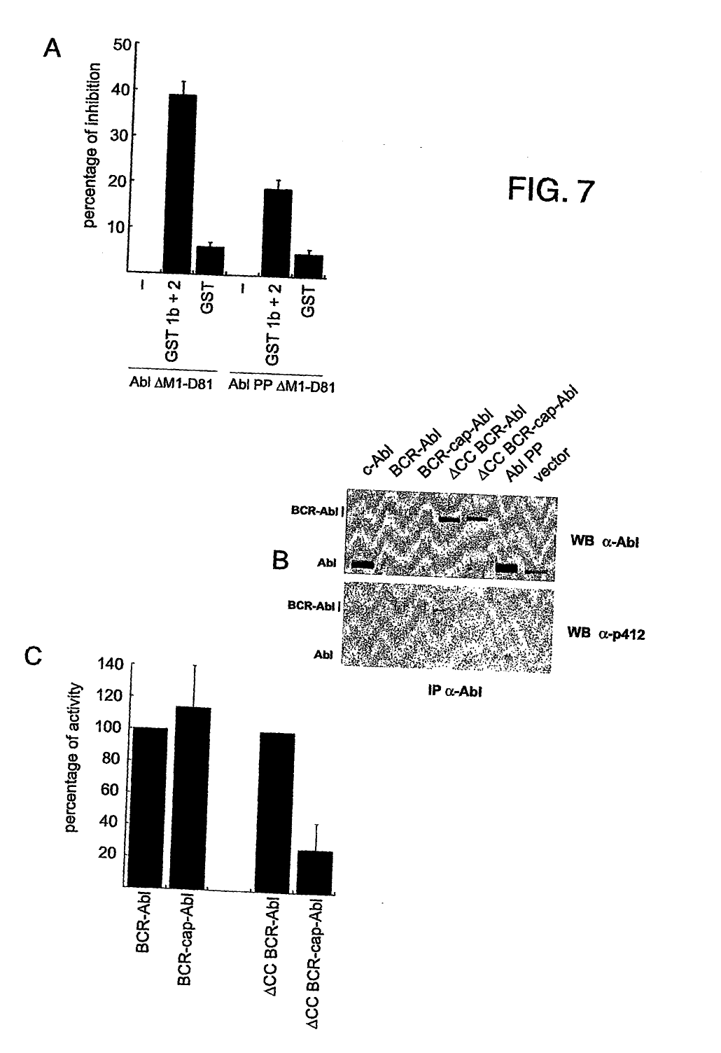

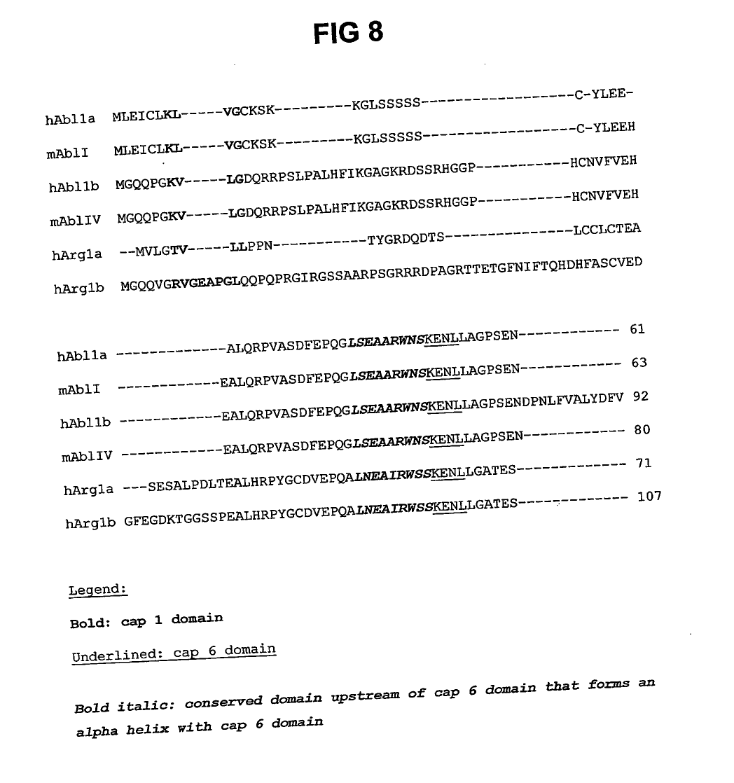

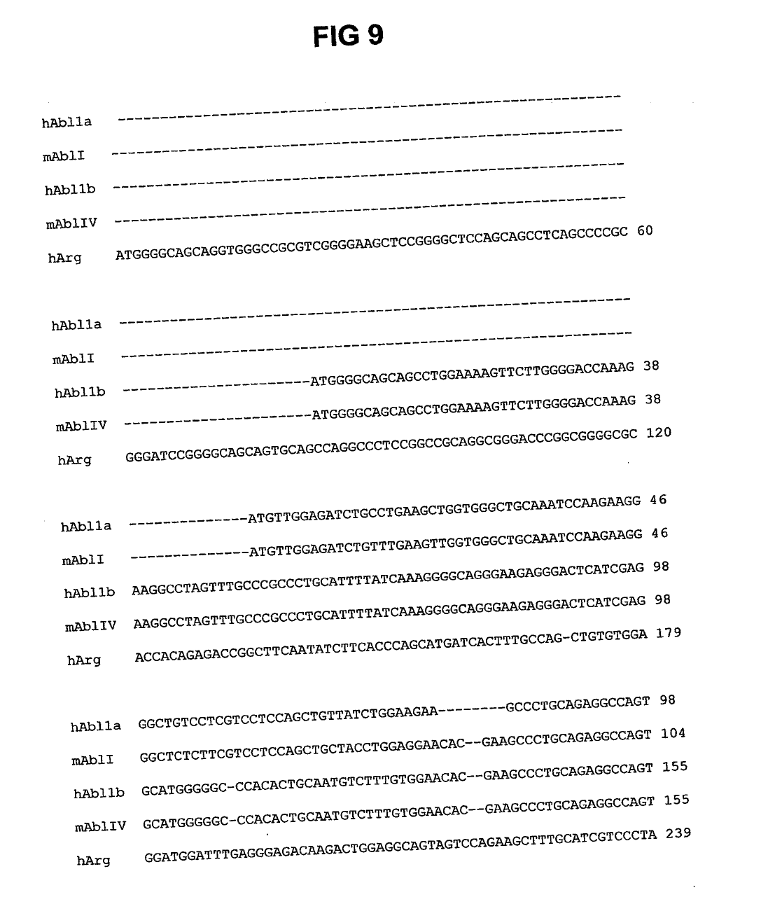

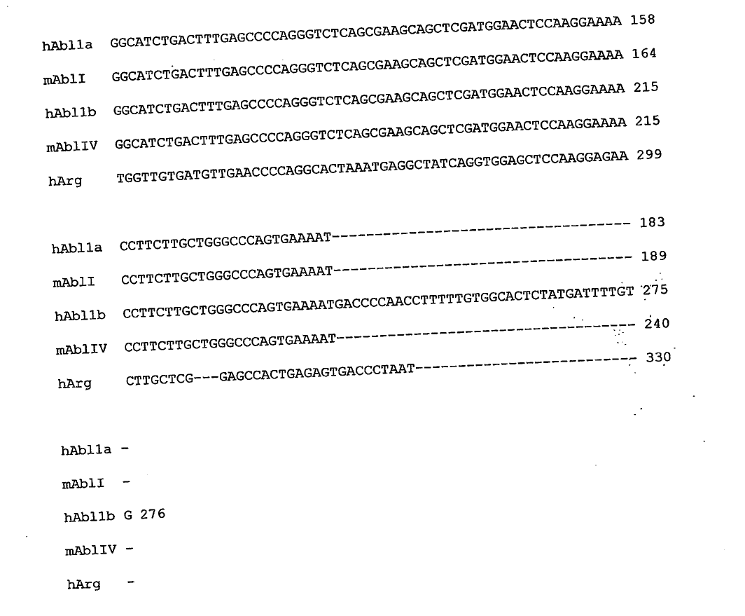

c-Abl and Abl-PP, a deregulated form in which two prolines in the putative intramolecular SH3-binding region connecting the SH2 domain to the catalytic domain are mutated (P242E/P249E), were expressed in wheat germ extract. Analysis of total cellular proteins as well as of immunoprecitated c-Abl revealed that no tyrosine phosphorylation could be detected when c-Abl is expressed (FIG. 1A). However, expression of Abl-PP resulted in the phosphorylation of c-Abl itself and of a number of endogenous proteins, showing that c-Abl is regulated in extracts of plant cells.

We have previously reported that c-Abl and an SH3 domain deletion form of Abl were equally active and toxic in the yeast S. pombe (Walkenhorst et al., 1996). We addressed again regulation of c-Abl in S. pombe, this time using an inducible promoter of much weaker activity than the one used previously. In this case, SH3 domain-dependent regulation was observed at early induction time points, as judged by a strong difference in tyrosine phosphorylation of cellular proteins between c-Abl and Abl-PP-containing yeast cells (FIG. 1B). After 24 hours, however when more c-Abl protein had accumulated, this difference was abolished. Thus, c-Abl is regulated in S. pombe, but regulation is sensitive to the levels of protein expression.

Regulation of c-Abl In Vitro

We purified c-Abl and Abl-PP from transfected human embryonic kidney (HEK) 293 cells (FIG. 1C). After a purification procedure that included a high-salt wash step, the Coomassie-blue-stained patterns of c-Abl and Abl-PP proteins were very similar, indicating that any protein(s) that may be responsible for the regulation of c-Abl in vivo does not co-purify in detectable amounts or is either too large or too small to be detected. Constitutively active Abl-PP is strongly phosphorylated on tyrosine and serine residues and thus migrates slightly more slowly than c-Abl (FIG. 1C, right panel and Dorey et al., 1999). To determine the activity of the purified Abl proteins we tested their in vitro kinase activities using GST-Crk as a substrate. A more than six-fold difference in activity between c-Abl and Abl-PP was measured (FIG. 1C, left panel). We also tested a mutant in which the two prolines in the SH2-CD linker are mutated to alanine (Abl-P242A/P249A), to find that it was activated to a similar extent as Abl-P242E/P249E (data not shown). To confirm that the difference in activity involved SH3-domain-dependent regulation, we tested various other mutants, including point mutations within the SH3 domain, v-Abl and a mutant in which a putative intramolecular salt bridge between the SH3 and catalytic domain is disrupted (Abl K313E Barilá and Superti-Furga, 1998). All deregulated forms were 7 to 16 times more active than wild-type c-Abl in in vitro kinase assays (data not shown). Similar results were also obtained with other anti-Abl antibodies and other substrates (data not shown). These results show that other cellular proteins are not necessary to maintain c-Abl in a state of catalytic inhibition.

Although it is unclear why it has been difficult to detect such differences previously (Pendergast et al., 1991; Mayer and Baltimore, 1994; Dorey et al., 1999) and several parameters may exert an influence on c-Abl regulation in vitro, we found that the nature of the divalent salt used in the kinase activity reaction has a profound effect on the catalytic activity. While the use of MgCl₂ at 10 mM concentration allows the detection of significant differences in catalytic activity of c-Abl versus Abl PP, the same samples assayed in the presence of 10 mM MnCl₂ resulted in no detectable difference in catalytic activity (data not shown).

Minimal Region Required for c-Abl Regulation

The results on c-Abl regulation in vitro prompted an investigation of the minimal part of the protein required for regulation. The “last exon region” has previously been implicated in c-Abl regulation and represents the binding site for several cellular proteins, including F- and G-actin (Goga et al., 1993; McWhirter and Wang, 1993; Van Etten et al., 1994; Woodring et al., 2001). We constructed a deletion mutant lacking the last exon region of c-Abl (Abl M1-K531, FIG. 2A). We also engineered a mutant lacking, additionally, the N-terminal amino acids preceding the SH3 domain, known to be dispensable for regulation of Src family kinases (Abl P82-K531, FIG. 2A).

After transient expression in HEK293 cells, cellular proteins were analyzed for phosphotyrosine content as a measure of in vivo protein activity and Abl proteins were immunoprecipitated to test for tyrosine phosphorylation as well as catalytic activity in vitro. Deletion of the last exon region (Abl M1-K531) did not lead to activation of Abl (FIG. 2B-C), but the additional double proline mutation in the SH2-catalytic domain linker (Abl-PP M1-K531) resulted in strong activation. These results suggest that the SH3-domain-dependent regulation is operational within the short form of Abl. Deletion of the last exon region abolished the ability of Abl to phosphorylate GST-Crk, but not GST-Jun, probably due to the loss of the Crk binding site (Ren et al., 1994).

Abl P82-K531 showed elevated tyrosine phosphorylation levels in total extracts and was highly active in vitro after immunoprecipitation when compared to its counterpart containing the normal N-terminal sequences (Abl M1-K531; FIG. 2). This suggests an involvement of the first 81 residues of the c-Abl protein in regulation which is in sharp contrast to Src family kinases, where residues N-terminal to the SH3 domain are dispensable for intramolecular regulation (Koegl et al., 1995; Sicheri and Kuriyan, 1997). Taken together, our results demonstrate that the minimal region necessary and sufficient for the regulation of c-Abl comprises the SH3, SH2, catalytic domain as well as the N-terminal residues.

Regulation of c-Abl by its N-Terminus

To obtain functional insight into the role of the N-terminal region in the regulation of full-length c-Abl, we constructed additional mutants and analyzed their catalytic activity in vitro and in vivo (FIG. 3). As expected from the results with the short Src-like form (Abl P82-K531), Abl ΔM1-D81 was strongly active also in the presence of the last exon region and lead to efficient phosphorylation of cellular proteins, Abl autophosphorylation and high catalytic activity in an in vitro kinase assay (FIG. 3B-C). Introduction of the deregulating mutation in the SH2-catalytic domain linker in Abl ΔM1-D81 (Abl-PP ΔM1-D81) had only a small further effect, suggesting that through deletion of the N-terminal 81 residues, Abl becomes fully deregulated. The effect of individually deleting the first 45 residues (exon 1b, Abl ΔM1-H45), or the following 36 residues (Abl AE46-D81) was less pronounced (FIG. 3B-C). Thus, the N-terminal amino acids encoded by the Abl type 1b exon and the first part of exon 2 are both required to keep c-Abl in its regulated state.

We also tested if the absence of this regulatory N-terminal region activated the oncogenic potential of c-Abl. In focus-formation assays using NIH3T3-P cells Abl ΔE46-D81 was a reliable oncogene, reaching a transformation efficiency comparable to point mutations in the SH3 domain (Table 1; Barilá and Superti-Furga, 1998).

The N-Terminal Region Interacts with c-Abl in trans

We tested the ability of the N-terminus of Abl to interact with the rest of c-Abl. GST fusion proteins containing the first exon (amino acids 1-45; GST 1b), first and part of the second exon (amino acids 1-80; GST 1b+2) and only second exon of c-Abl type 1b (amino acids 46-80; GST 2) were prepared and used to pull-down transiently expressed c-Abl, Abl ΔM1-D81 and Abl-PP ΔM1-D81 proteins in extracts derived from transfected HEK293 cells (FIG. 4A). Since in the ABL1 gene two alternative first exons are spliced to give rise to c-Abl type 1a and 1b (Shtivelman et al., 1986) we also tested similar constructs of c-Abl type 1a (amino acids 1-26; GST 1a and 1-61; GST 1a+2). All five Abl GST-fusion proteins were able to pull down Abl ΔM1-D81, suggesting a direct interaction between the Abl N-terminal amino acids and Abl ΔM1-D81 in trans. In contrast, c-Abl was not bound by the GST-fusion proteins, suggesting that the binding site in c-Abl is “covered” by the N-terminus in cis. Abl-PP ΔM1-D81 bound the GST-fusion proteins only weakly. Thus, the N-terminal region appears to bind the rest of Abl in a conformation-dependent manner.

To define the region in c-Abl to which the N-terminal region binds, GST fusion proteins of the first exon type 1a, type 1b and of the second exon were tested for their ability to pull down transiently expressed c-Abl proteins (FIG. 4B), including the SH3-SH2-linker-catalytic domain portion (SH3-SH2-CD), the SH3-SH2 domains or of the catalytic domain only (CD). The SH3-SH2-CD protein was bound by all three GST fusion proteins. The catalytic domain was bound by first exons 1a and 1b only, while the second exon bound preferentially to the portion of Abl containing of the SH3 and SH2 domains. Thus, different parts of the N-terminal region appears to bind different parts of c-Abl, as if clamping the protein together. Because of its position at the N-terminus of c-Abl, we refer to this region as the “cap”.

Mutational Analysis of the N-Terminal Region

To understand better the nature of the interaction between the “cap” and the rest of Abl, we performed alanine-scanning mutagenesis of groups of four, mostly polar, residues, in the common second exon region and that appeared conserved between the 1a and 1b alternative exons (FIG. 5A, cap 1-6). After transient expression in HEK293 cells, Abl proteins were immunoprecipitated to test for tyrosine phosphorylation content as well as for catalytic activity in vitro. The cap 1 and cap 6 mutants were phosphorylated and exhibited an increased catalytic activity (FIG. 5B). To establish a possible correlation between the role of cap 1 and cap 6 residues in regulation and their binding properties, GST fusion proteins were prepared of Abl exon 1b with the cap 1 mutation (GST 1b cap 1) and exon 2 with the cap 6 mutation (GST 2 cap 6) and used to pull down transiently expressed SH3-SH2-CD and CD Abl proteins from HEK293 cell extracts (FIG. 5C). Both mutation of cap 1 and cap 6 residues prevented binding to the SH3-SH2-CD protein, and the relatively strong binding of exon 1b to the catalytic domain was strongly reduced by mutation of cap 1 residues. These results show that both conserved residues of exon 1 and residues of exon 2 are required for the binding to Abl and its regulation.

The N-Terminal Region is Required to Maintain the Regulated State

The N-terminal region may only be required to assemble the regulatory apparatus, possibly during folding, and then become dispensable. Alternatively, the N-terminal region may target c-Abl to particular subcellular sites and only indirectly affect c-Abl regulation. To rule out these possibilities, we took advantage of the presence of residues resembling the cleavage site for the highly specific Tobacco-Etch-Virus (TEV) protease roughly at the boundary between the N-terminus and the SH3 domain. We engineered a perfect TEV site by mutating four residues to obtain Abl-TEV. The TEV cleavage would occur seven residues upstream of Phe85, representing the beginning of the PA strand of the SH3 domain (FIG. 6A; Musacchio et al., 1994). Mutation of the four residues required to engineer the TEV site did not affect c-Abl regulation (FIG. 6B). We transfected HEK293 cells with Abl-TEV as well as with control Abl constructs. Abl proteins were immunoprecipitated and treated or not with TEV protease. TEV treatment caused the appearance of a faster-migrating form of Abl-TEV but not of c-Abl (FIG. 6B). Kinase assays performed in parallel revealed a TEV-dependent increase in catalytic activity only in Abl-TEV and not in c-Abl or Abl ΔM1-D81 (FIG. 6B). The extensive washes of the immunoprecipitated protein likely removed the cleaved N-terminal portion. Thus, cleaving the N-terminal 77 residues of c-Abl in vitro leads to its activation. We conclude that the N-terminal residues are necessary to maintain the inhibited state of c-Abl and that they have a critical role in its regulation.

The N-Terminal Region Inhibits Abl In Vitro and Restores Regulation of BCR-ABL

To test whether the interactions of the “cap” are sufficient to restore Abl regulation in vitro, we purified a GST fusion protein of the N-terminal region of type 1b Abl (GST 1b+2) and incubated it with immunoprecipitated Abl ΔM1-D81 and with its counterpart bearing the additional PP mutation in the linker (FIG. 7A). While the GST control protein had no effect on catalytic activity, GST 1b+2 inhibited Abl ΔM1-D81 activity roughly 40%. A significant but reduced level of inhibition was achieved also with Abl-PP ΔM1-D81. This result could indicate a potential direct effect on Abl's catalytic activity, rather than on mere “regulation” and could also reflect the residual binding of GST 1b+2 to Abl-PP ΔM1-D81 observed previously (FIG. 4).

BCR-ABL fusion proteins invariably miss the first exon sequences. If Abl's “cap” region is so powerful that it can re-regulate even active forms of Abl (such as Abl ΔM1-D81), the missing first exon may restore regulation of BCR-ABL if reintroduced in the molecule between the BCR sequences and the beginning of the second exon (BCR-cap-Abl; FIG. 7B). In parallel to such constructs, we also tested versions in which the coiled-coil region of BCR-ABL, critically involved in dimerization, are missing (ΔCC BCR-cap-Abl; McWhirter et al., 1993). Expression in HEK293 cells was equally efficient for all BCR-ABL forms (data not shown). Immunoprecipitation was less efficient for BCR-ABL proteins than for the forms bearing the deletion of the coiled-coil region (FIG. 7B, upper panel). To monitor BCR-Abl's activity in the cell, we chose to use antibodies specific for tyrosine 412 in the activation loop, likely to reflect the state of activity better than anti-phosphotyrosine, since BCR-ABL is tyrosine phosphorylated also at other sites that may not be directly dependent on catalytic activity (Pendergast et al., 1993a; Pendergast et al., 1993b). As expected, if compared to the levels of immunoprecipitated protein, the forms bearing the coiled-coil deletion were less phosphorylated at Tyr412, reflecting the importance of dimerization for BCR-ABL activity (FIG. 7B; McWhirter et al., 1993; Smith and Van Etten, 2001). Introduction of the first exon sequences did not have a measurable effect on Tyr412 phosphorylation of BCR-ABL but caused a dramatic reduction in the activity of BCR-Abl bearing the coiled-coil deletion. To test whether this decrease in activation loop phosphorylation also reflected a reduced catalytic activity in vitro, we performed kinase assays with the different immunoprecipitated BCR-Abl proteins. The cap-bearing version of ACC BCR-Abl showed about 25% of the activity of its normal counterpart (FIG. 7C). Together, these data show that the first exon region is capable of restoring regulation to a dimerization-deficient BCR-Abl, confirm the dominant features of dimerization in overriding the natural regulation of Abl, and suggest that loss of the first exon may contribute to deregulation of BCR-ABL.

2) Discussion

The data presented here demonstrate regulation of purified c-Abl in vitro as well as regulation in non-animal expression systems. Regulation of c-Abl activity is thus an intrinsic property and does not require a particular cellular inhibitor. An intramolecular sandwich involving the SH3 domain, the linker between the SH2 and catalytic domain and the catalytic domain itself has been proposed to regulate the activity of c-Abl as it does in Src family kinases (Barilá and Superti-Furga, 1998). In Src family kinases, however, the assistance of the C-terminal “tail” region is essential. What structure in c-Abl substitutes for the C-terminal tail of Src? The last exon region of c-Abl bears nuclear import and nuclear export signals on top of binding sites for a variety of cellular proteins including actin, Crk, Nck, p53. There is genetic evidence that it plays a role in the regulation of c-Abl in cells (Goga et al., 1993; Woodring et al., 2001). We show however, that the last exon region of c-Abl is totally dispensable for the SH3 domain-dependent regulation of catalytic activity as defined here and measured in vitro.

If the last exon region is dispensable, what is minimally required? In addition to the SH3, SH2 and catalytic domains, the Src-like “core”, we found an unexpected critical role for the first 81 residues of the c-Abl protein. In this respect, c-Abl differs to Src family kinases, where residues N-terminal to the SH3 domain are dispensable for intramolecular regulation (Koegl et al., 1995; Sicheri and Kuriyan, 1997). An immediate suggestion concerning a regulatory role of the N-terminus comes from the fact that in the ABL1 gene, two alternative first exons are spliced to give rise to c-Abl type 1a or 1b (Shtivelman et al., 1986). c-Abl 1a is 19 amino acids shorter, is spliced much less frequently and does not include a myristoylation signal. There is no evidence in the literature that would indicate different levels of activity between the two forms. Transgenes encoding both type I and type IV c-Abl proteins rescue the lethality of c-abl mutant mice (Hardin et al., 1996). Our attempts to express Abl bearing the type 1a exon in cells failed, consistent with the report of others (Van Etten, 1999), but when translated in reticulocyte lysates, type 1a Abl is regulated as well as type 1b (K. Dorey, unpublished results). Early reports had addressed the role of the N-terminus in the regulation of c-Abl (Franz et al., 1989; Jackson and Baltimore, 1989; Wang, 1988), but the results were not sufficiently conclusive, and attention focused on the discovery and function of the adjacent SH3 domain, obscuring the role of the extreme N-terminus of c-Abl.

We suggest a novel model according to which c-Abl is regulated by a set of intramolecular interactions (FIG. 6C). While Src family kinases have an interaction of their “tail” with their own SH2 domain that contributes critically to maintenance of the SH3 domain-dependent regulation, c-Abl has an N-terminal “cap” that serves an analogous function. This cap appears to bind at several portions “across” the molecule and stabilize the regulated, inhibited conformation. According to our data, the “KV/LV/LG” motif (see cap 1 region in FIG. 5A), which is common to both type 1a and 1b exons and required for binding, must undergo relatively strong interactions with the catalytic domain. The first part of the common second exon does not interact with the catalytic domain but interacts with the SH3 and/or SH2 domains. FIG. 6C shows the cap as if binding to the “north-face” merely for graphic convenience. In fact, because the two regions known to be critical for binding (the cap 1 and cap 6 residues) are spaced differently in the 1a and 1b forms of Abl, some of the 19 additional residues of the type 1b cap may “loop out” from whatever is the minimal “bridge” from the SH3 to the catalytic domain.

The experiment with the engineered form of c-Abl in which the presence of the N-terminal region is removed in vitro, has shown that the N-terminus is required to maintain and not merely to assemble the regulated conformation. This is also confirmed by the ability of cap sequences to inhibit cap-less Abl in vitro. This mechanism may be exploited by cellular proteins that inhibit c-Abl in trans.

In general, cellular proteins may either inhibit or activate c-Abl by favoring or displacing any of the several critical intramolecular interactions. For example numerous proteins bind the Abl SH3 domain (reviewed in Van Etten, 1999). Moreover, an interdependence of the intramolecular interactions and catalytic activity, as in Src family kinases, seems highly probable (Gonfloni et al., 2000). In this view, phosphorylation of the activation loop with its conformational effects on the catalytic domain and the SH3 and cap-mediated inhibitory intramolecular interactions antagonize each other. The degree of catalytic activity and the degree of SH3 domain availability are the net result of these opposing forces. In fact, the PTP-PEST tyrosine phosphatase, dephosphorylating the activation loop, acts as an inhibitor of c-Abl (Cong et al., 2000), while Src family kinases and Abl itself act as activators by causing phosphorylation (Plattner et al., 1999; Brasher and Van Etten, 2000; Dorey et al., 2001).

We believe that the cap represents the missing link in c-Abl's intramolecular regulation. The first exon region is lacking in all of the different fusion proteins formed with BCR or TEL resulting from chromosomal translocations and also in v-Abl. The dimerization properties of BCR and TEL are thought to induce cross-phosphorylation and activation of the catalytic domains by induced proximity and thus represent critical “gain-of-function” alterations of c-Abl (Golub et al., 1996; McWhirter et al., 1993; Smith and Van Etten, 2001). Moreover, signaling properties in the BCR portion of BCR-ABL are known to be critical for transformation (reviewed in Sawyers, 1992). Our data suggest that the absence of the N-terminal cap in BCR-Abl (and in TEL-Abl and v-Abl), represents a “loss-of-function” alteration which contributes to the acquisition of constitutive tyrosine kinase activity in these oncogenic forms. Thus, the cap of c-Abl may represent what the C-terminal tail represents for c-Src. A crystal structure of c-Abl including the cap will be essential to elucidate the precise molecular mechanism of regulation, and future work will address how the cap may modulate c-Abl differentially in the different splice variants and in the context of cellular signaling networks.

3) Experimental Procedures

DNA Constructs

pSGT vector and pSGT-Abl constructs were previously described (Barilá and Superti-Furga, 1998). Abl M1-K531, Abl-PP M1-K531 and Abl P82-K531 were obtained by PCR with c-Abl type 1b as template and subcloned in pSGT vector. For preparation of Abl ΔM1-H45, Abl AE46-D81, Abl ΔM1-D81 and Abl-PP ΔM1-D81, pSGT-c-Abl or pSGT-Abl-PP were digested with EcoRI and KpnI and the released N-terminal fragment was replaced by a PCR product containing the desired deletion. Point mutations and cap mutants (cap 1-6) were obtained using the quick-change site directed mutagenesis kit (Stratagene) and pSGT-c-Abl type 1b DNA as template. All mutagenesis constructs were confirmed by sequencing. The SH3-SH2-CD (N80-K531), SH3-SH2 (N80-P235) or CD (D252-K531) Abl protein parts were amplified using bAbl as a template and subcloned into a CMV driven vector containing a HA tag (described in Barilá et al., 2000).

The p210 BCR-Abl is a kind gift from Owen Witte. pSGT BCR-Abl and pSGT □CC BCR-Abl have been reconstituted by amplifying respectively M1-F1059 and L61-F1059. The resulting BCR-Abl fragments were digested EcoRI/Kpn I (a unique internal site in Abl) and subdloned into pSGT hAbl backbone. To generate pSGT BCR-cap-Abl and pSGT ΔCC BCR-cap-Abl, BCR (M1-S927) or ΔCC BCR (L61-S927) have been amplified using p210 as a template. The PCR products were digested Eco RI/Not I and subcloned into pSGT hAbl. All PCR products were sequenced.

Expression of Abl in Wheat Germ Extract and S. pombe

c-Abl and Abl-PP RNA were prepared as described (Dorey et al., 1999) and used for expression of protein in wheat germ extract using a commercial translation system (Promega). pSGT-c-Abl, ASH3-Abl and Abl-PP (Barilá and Superti-Furga, 1998) were subcloned to the yeast pRWP vector, a derivative of pRSP (Superti-Furga et al., 1993) containing a mutation of the nmt1 promoter making it approximately ten times weaker (Basi et al., 1993), and transformed to S. pombe strain SP200 (Superti-Furga et al., 1993). Expression of Abl protein was induced by removal of thiamine as described (Walkenhorst et al., 1996) and yeast cellular protein extracts were made by boiling pelleted yeast cells in SDS-sample buffer 16 and 24 hours after induction.

Transfection and Immunoprecipitation

HEK293 cells were cultured in Dulbecco's Modified Eagle's Medium supplemented with 10% fetal calf serum. Cells were transfected with pSGT-Abl DNAs using the calcium phosphate method. Forty hours after transfection cells were lysed in IP buffer (50 mM Tris-HCl pH 7.5, 150 mM NaCl, 1% NP-40, 5 mM EDTA, 5 mM EGTA, 25 mM NaF, 1 mM orthovanadate, 1 mM PMSF, 10 μg/ml TPCK, 5 μg/ml TLCK, 1 μg/ml leupeptin, 1 μg/ml aprotinin, 10 μg/ml soybean trypsin inhibitor) and insoluble material was removed by centrifugation (10′ 13,000 rpm). Abl protein was immunoprecipitated from total cell lysates in IP buffer using anti-Abl antibody (Ab-3, Oncogene Science or K12, Santa Cruz). Immune complexes were recovered using protein G-Sepharose beads, assayed for Abl catalytic activity and analyzed by SDS-PAGE followed by anti-Abl (Ab-3 or K12) and anti-phosphotyrosine (4G10, Upstate Biotechnology) immunoblotting. Anti-p412 antibodies that recognize specifically Abl when phosphorylated in the activation loop were a kind gift from Dr J. Wu (Cell Signaling Technology (CST) Inc, Beverly, Mass.).

Large-Scale Purification of Abl Protein

c-Abl and Abl-PP protein were expressed by transient transfection in HEK293 cells. Forty hours after transfection cells were lysed in IP buffer and insoluble material was removed by centrifugation at 100.000g for 1 h. Cell lysates were incubated with anti-Abl antibodies (Ab-3, Oncogene Science) covalently coupled to protein G-Sepharose beads. Beads were washed with IP buffer followed by phosphate buffer (50 mM NaPhosphate pH 6.3, 0.1% Triton-X100, 500 mM NaCl). Bound proteins were used for Abl kinase assay directly or eluted by pH shock (50 mM glycine pH 2.5, 0.1% Triton-X100, 150 mM NaCl) and run on a 4-15% gradient SDS-PAGE gel (Biorad). Abl and co-purifying proteins were detected by brilliant blue colloidal Coomassie staining (Sigma).

Abl Kinase Assay

The catalytic activity of purified Abl protein bound to protein G-Sepharose beads was determined as follows. Beads were washed three times with IP buffer, two times with IP buffer without NaCl and two times with kinase assay buffer (20 mM Tris-HCl pH 7.5, 10 mM MgCl₂, 1 mM DTT). Twenty μl linase assay mix (1 μg GST-Crk121-226 (Dorey et al., 1999), 0.5 μl [γ-³²P]-ATP (Amersham), 0.1 mM ATP in kinase assay buffer) was added and the mixture was incubated at room temperature for 10 and 20 minutes. The kinase reaction was stopped by adding SDS-sample buffer and analyzed by SDS-PAGE. Quantification of the reaction was done by cutting the desired bands from gel followed by liquid scintillation counting and/or phosphoimager analysis. For the in vitro inhibition of Abl, GST or GST 1b+2 protein were bacterially expressed, purified and dialysed against kinase assay buffer. Equal quantities of purified proteins were added directly to the kinase assay mixture, samples were incubated for 2 hours at 4° C. and then assayed as above.

Focus Formation Assay

Abl DNAs were subcloned from pSGT to pSLX vector (Renshaw et al., 1995) by BamHI digestion. Focus formation assay was performed in NIH3T3-P cells (Renshaw et al., 1992) using 0.5 μg of pSLX-Abl DNA essentially as described (Barila and Superti-Furga, 1998; Renshaw et al., 1995). Percentage of foci was calculated by dividing the number of foci by the number of neomycin resistant colonies of each construct.

In Vitro Binding Assay

DNA fragments corresponding to c-Abl amino acids, M1-H45 (1st exon type 1b), E46-N80 (beginning of the 2nd exon until the start of the SH3 domain) and M1-N80 (1 st exon type 1b+2nd exon) were amplified by PCR using c-Abl type 1b as template. M1-E26 (1st exon type 1a) and M1-N61 (1st exon type 1a+2nd exon) were generated using c-Abl type 1a as template. All PCR products were cloned in pGEX-2T vector. Bacterially produced Abl GST fusion proteins were pre-bound to glutathion-Sepharose beads and incubated with 2 mg of HEK293 cell lysate containing c-Abl, Abl ΔM1-D81, Abl-PP ΔM1-D81 or HA-tagged Abl protein parts (SH3-SH2-CD, SH3-SH2 or CD) for 3 hours at 4° C. Bound proteins were analyzed by SDS-PAGE followed by anti-Abl or anti-HA (12CA5, Boehringer Mannheim) immunoblotting.

TEV Cleavage of Abl Protein

An artificial TEV cleavage site was engineered in c-Abl just before the SH3 domain using the quick-change site directed mutagenesis kit (Stratagene). c-Abl amino acids 74-77 (LAGP) were replaced by YFQG introducing a perfect TEV consensus site, giving rise to Abl-TEV. After expression of Abl-TEV in HEK293 cells and immunoprecipitation using anti-Abl antibodies (Ab-3, Oncogene Science), the immune complexes bound to protein G-Sepharose beads were incubated for 2 hours at 16° C. in 10 mM Tris pH 8.0, 100 mM NaCl, 0.1% Igepal, 0.5 mM EDTA with or without 10 units of TEV enme (Gibco). Subsequently, beads were processed as described for Abl kinase assay.

TABLE 1
Transformation potential of Abl ΔE46-D81

| | Neo colonies | Foci in 5% CS | Transformation |
|---|---|---|---|
| | (mean ± SD) | (mean ± SD) | efficiency (%) |
| PSLX c-Abl | 5955 ± 505 | 13 ± 3 | 0.2 |
| PSLX Abl ΔE46-D81 | 2170 ± 170 | 215 ± 35 | 9.9 |
| PSLX Abl-PP | 2135 ± 185 | 615 ± 125 | 28.8 |

NIH3T3-P cells transfected with the indicated Abl constructs in vector pSLX were grown for 16 days in DMEM containing 5% calf serum to allow the formation of foci. Duplicate dishes were grown under neomycin selection to determine the transfection efficiency. The transformation efficiency was calculated by dividing the number of foci by the number of neomycin resistant colonies of each construct. Results shown are the averages of two experiments done in duplicate.

### REFERENCES

Ausubel et al, Current Protocols in Molecular Biology, Wiley Interscience, New York, 1991;
Barilá, D., Mangano, R., Gonfloni, S., Kretzschmar, J., Moro, M., Bohmaim, D., and Superti-Furga, G. (2000). A nuclear tyrosine phosphorylation circuit: c-Jun as an activator and substrate of c-Abl and JNK. EMBO J. 19, 273-281.
Jackson P, Baltimore D. (1989) N-terminal mutations activate the leukemogenic potential of the myristoylated form of c-abl. EMBO J. 8(2):449-56
Barilá, D., and Superti-Furga, G. (1998). An intramolecular SH3-domain interaction regulates c-Abl activity. Nat. Genet. 18, 280-282.
Basi, G., Schmid, E., and Maundrell, K. (1993). TATA box mutations in the Schizosaccharomyces pombe nmt1 promoter affect transcription efficiency but not the transcription start point or thiamine repressibility. Gene 123, 131-136.
Brasher, B. B., and Van Etten, R. A. (2000). c-Abl has high intrinsic tyrosine kinase activity that is stimulated by mutation of the src homology 3 domain and by autophosphorylation at two distinct regulatory tyrosines. J. Biol. Chem. 275, 35631-35637.
Cong, F., Spencer, S., Cote, J. F., Wu, Y., Tremblay, M. L., Lasky, L. A., and Goff, S. P.(2000). Cytoskeletal protein PSTPIPI directs the PEST-type protein tyrosine phosphatase to the c-Abl kinase to mediate Abl dephosphorylation. Molecular Cell 6, 1413-1423.
Dorey, K., Barila, D., Gavin, A. C., Nebreda, A. R., and Superti-Furga, G. (1999). Regulation of human c-Abl tyrosine kinase activity in Xenopus oocytes and acceleration of progesterone-induced G2/M transition by oncogenic forms. Biol. Chem. 380, 223-230.
Dorey, K., Engen, J. R., Kretzchmar, J., Wilm, M., Neubauer, G., Schindler, T., and Superti-Furga, G. (2001). Phosphorylation and structure-based functional studies reveal multiple roles for the activation loop of the c-Abl tyrosine kinase. Oncogene, in press.
Druker, B. J., Tamura, S., Buchdunger, E., Ohno, S., Segal, G. M., Fannin, S., Zimmerman, J., and Lydon, N. B. (1996). Effects of a selective inhibitor of the Abl tyrosine kinase on the growth of Bcr-Abl positive cells. Nat. Med. 2, 561-566.
Fernandez J. M. & Hoeffler J. P. Gene expression systems. Using nature for the art of expression ed. Academic Press, San Diego, London, Boston, New York, Sydney, Tokyo, Toronto, 1998.
Franz, W. M., Berger, P., and Wang, J. Y. J. (1989). Deletion of an N-terminal regulatory domain of the c-Abl tyrosine kinase activates its oncogenic potential. EMBO J. 8, 137-147.
Goga, A., McLaughlin, J., Pendergast, A. M., Parmar, K., Muller, A., Rosenberg, N., and Witte, O. N. (1993). Oncogenic activation of c-Abl by mutation within its last exon. Mol. Cell. Biol. 13, 4967-4975.
Golub, T. R., Goga, A., Barker, G. F., Afar, D. E. H., McLaughlin, J., Bohlander, S. K., Rowley, J. D., Witte, O. N., and Gilliland, D. G. (1996). Oligomerization of the Abl tyrosine kinase by the Ets protein TEL in human leukemia. Mol. Cell. Biol. 16, 4107-4116.
Gonfloni, S., Weijland, A., Kretzschmar, J., and Superti-Furga, G. (2000). Crosstalk between the catalytic and regulatory domains allows bidirectional regulation of Src. Nat. Struct. Biol. 7, 281-286.
Gorre M E, Mohammed M, Ellwood K, Hsu N, Paquette R, Rao P N, Sawyers C L. (2001) Clinical resistance to STI-571 cancer therapy caused by BCR-ABL gene mutation or amplification Science 293(5531):876-80
Hardin, J. D., Boast, S., Mendelsohn, M., de los Santos, K., and Goff, S. P. (1996). Transgenes encoding both type I and type IV c-abl proteins rescue the lethality of c-abl mutant mice. Oncogene 12, 2669-2677.
Jackson, P., and Baltimore, D. (1989). N-terminal mutations activate the leukemogenic potential of the myristylated form of c-abl EMBO J. 8, 449-456.
Koegl, M., Courtneidge, S. A., and Superti-Furga, G. (1995). Structural requirements for the efficient regulation of the Src protein tyrosine kinase by Csk. Oncogene 11, 2317-2329.
Mayer, B. J., and Baltimore, D. (1994). Mutagenic analysis of the roles of SH2 and SH3 domains in regulation of the Abl tyrosine kinase. Mol. Cell. Biol. 14, 2883-2894.
McWhirter, J. R., Galasso, D. L., and Wang, J. Y. (1993). A coiled-coil oligomerization domain of Bcr is essential for the transforming function of Bcr-Abl oncoproteins. Mol. Cell. Biol. 13, 7587-7595.
McWhirter, J. R., and Wang, J. Y. (1993). An actin-binding function contributes to transformation by the Bcr-Abl oncoprotein of Philadelphia chromosome-positive human leukemias. EMBO J. 12, 1533-1546.
Musacchio, A., Saraste, M., and Wilmanns. M. (1994). High-resolution crystal structures of tyrosine kinase SH3 domains complexed with proline-rich peptides. Structural Biol. 1, 546-551.
Pendergast, A. M., Gishizky, M. L., Havlik, M. H., and Witte, O. N. (1993a). SH1 domain autophosphorylation of P210 BCR/ABL is required for transformation but not growth factor independence. Mol. Cell. Biol. 13, 1728-1736.
Pendergast, A. M., Muller, A. J., Havlik, M. E., Clark, R., McCormick, F., and Witte, O. N. (1991). Evidence for regulation of the human ABL tyrosine kinase by a cellular inhibitor. Proc. Natl. Acad. Aci. USA 88, 5927-5931.
Pendergast, A. M., Quilliam, L. A., Cripe, L. D., Bassing, C. H., Dai, Z., Li, N., Batzer, A., Rabun, K. M., Der, C. J., Schlessinger, J., and Gishizky, M. L. (1993b). BCR-ABL-induced oncogenesis is mediated by direct interaction with the SH2 domain of the GRB-2 adaptor protein. Cell 75, 175-185.
Plattner, R., Kadlec, L., DeMali, K. A., Kazlauskas, A., and Pendergast, A. M. (1999). c-Abl is activated by growth factors and Src family kinases and has a role in the cellular response to PDGF. Genes & Dev. 13, 2400-2411.
Prasher et al, (1995), Trends in Genetics, 11(8), 320
Raitano, A. B., Whang, Y. E., and Sawyers, C. L. (1997). Signal transduction by wild-type and leukemogenic Abl proteins. Biochim. Biophys. Acta 1333, F201-F216.
Ren, R., Ye, Z.-S., and Baltimore, D. (1994). Abl protein-tyrosine kinase selects the Crk adapter as a substrate using SH3-binding sites. Genes & Dev. 8, 783-795.
Renshaw, M. W., Kipreos, E., Albrecht, M. R., and Wang, J. Y. J. (1992). Oncogenic v-Abl tyrosine kinase can inhibit or stimulate growh, depending on the cell context. EMBO J. 11, 3941-3951.
Renshaw, M. W., McWhirter, J. R., and Wang, J. Y. J. (1995). The human leukemia oncogene bcr-abl abrogates the anchorage requirement but not the growth factor requirement for proliferation. Mol. Cell. Biol. 15, 1286-1293.
Sambrook et al Molecular cloning: a laboratory manual New York: Cold Spring Harbour Laboratory Press, 2000;
Sawyers, C. L. (1992). The bcr-abl gene in chronic myelogenous leukemia Cancer Surv. 15, 37-51.
Schindler, T., Bornmann, W., Pellicena, P., Miller, W. T., Clarkson, B., and Kuriyan, J. (2000). Structural mechanism for STI-571 inhibition of abelson tyrosine kinase. Science 289, 1938-1942.
Shtivelman, E., Lifshitz, B., Gale, R. P., Roe, B. A., and Canani, E. (1986). Alternative splicing of RNAs transcribed from the human abl gene and from the bcr-abl fused gene. Cell 47, 277-284.
Sicheri, F., and Kuriyan, J. (1997). Structures of Src-family tyrosine kinases. Curr. Opin. Struct. Biol. 7, 777-785.
Smith, K. M., and Van Etten, R. A. (2001). Activation of c-Abl kinase activity and transformation by a chemical inducer of dimerization. J. Biol. Chem. 276, 24372-24379. Spector et al Cells, a laboratory manual; Cold Spring Harbour Laboratory Press
Superti-Furga, G., and Courtneidge, S. A. (1995). Structure-function relationships in Src family and related protein tyrosine kinases. Bioessays 17, 321-330.
Superti-Furga, G., Fumagalli, S., Koegi, M., Courtneidge, S. A., and Draetta, G. (1993). Csk inhibition of Src activity requires both the SH2 and SH3 domains of Src. EMBO J. 12, 2625-2634.
Taagepera, S., McDonald, D., Loeb, J. E., Whitaker, L. L., McElroy, A. K., Wang, J. Y., and Hope, T. J. (1998). Nuclear-cytoplasmic shuttling of C-ABL tyrosine kinase. Proc. Natl. Acad. Sci. USA 95, 7457-7462.
Thiesing, J. T., Ohno-Jones, S., Kolibaba, K. S., and Druker, B. J. (2000). Efficacy of STI571, an abl tyrosine kinase inhibitor, in conjunction with other antileukemic agents against bcr-abl-positive cells. Blood 96, 3195-3199.
Van Etten, R. A. (1999). Cycling, stressed-out and nervous: cellular functions of c-Abl. Trends Cell Biol. 9, 179-186.
Van Etten, R. A., Jackson, P. K., Baltimore, D., Sanders, M. C., Matsudaira, P. T., and Janmey, P. A. (1994). The COOH-terminus of the c-Abl tyrosine kinase contains distinct F- and G-actin binding domains with bundling activity. J. Cell. Biol. 124, 325-340.
Vigneri, P., and Wang, J. Y. (2001). Induction of apoptosis in chronic myelogenous leukemia cells through nuclear entrapment of BCR-ABL tyrosine kinase. Nat. Med. 7, 228-234.
Walkenhorst, J., Goga, A., Witte, O. N., and Superti-Furga, G. (1996). Analysis of human c-Abl tyrosine kinase activity and regulation in S. pombe. Oncogene 12, 1513-1520.
Wang, J. Y. (2000). Regulation of cell death by the abl tyrosine kinase. Oncogene 19, 5643-50.
Wang, J. Y. J. (1988). Negative Regulation of c-abl tyrosine kinase by its variable N-terminal amino acids. Oncogene Res. 3, 293-298.
Woodring, P. J., Hunter, T., and Wang, J. Y. (2001). Inhibition of c-Abl tyrosine kinase by filamentous-actin. J. Biol. Chem. 276, 27104-27110.

1. A tyrosine kinase inhibitor protein consisting of the cap region of a c-Abl protein or a functional equivalent thereof.

2. A tyrosine kinase inhibitor protein according to claim 1 wherein the c-Abl protein is a mammalian c-Abl protein.

3. A tyrosine kinase inhibitor protein according to claim 2 wherein the c-Abl protein is human.

4. A tyrosine kinase inhibitor protein according to claim 3 wherein the c-Abl protein is type 1a c-Abl.

5. A tyrosine kinase inhibitor protein according to claim 3 wherein the c-Abl protein is type 1b c-Abl.

6. A tyrosine kinase inhibitor protein according to claim 4 consisting of amino acids 1-61 of type 1a c-Abl.

7. A tyrosine kinase inhibitor protein according to claim 5 consisting of amino acids 1-80 of Type 1b c-Abl.

8. A tyrosine kinase inhibitor protein according to claim 2 wherein the c-Abl protein is murine.

9. A tyrosine kinase inhibitor protein according to claim 8 wherein the c-Abl protein is type 1c-Abl.

10. A tyrosine kinase inhibitor protein according to claim 8 wherein the c-Abl protein is type IV c-Abl.

11. A tyrosine kinase inhibitor protein according to claim 9 consisting of amino acids 1-63 of type I c-Abl.

12. A tyrosine kinase inhibitor protein according to claim 10 consisting of amino acids 1-80 of type IV c-Abl.

13. A tyrosine kinase inhibitor protein or functional equivalent thereof according to claim 1 which inhibits a tyrosine kinase protein containing SH2 and SH3 domains.

14. A tyrosine kinase inhibitor protein or functional equivalent according to claim 13 which inhibits Abl, Src or Fyn.

15. A tyrosine kinase inhibitor protein or functional equivalent thereof according to claim 13 which inhibits an oncogenic form of said tyrosine kinase protein containing SH2 and SH3 domains.

16. A tyrosine kinase inhibitor protein or functional equivalent thereof according to claim 15 which inhibits an oncogenic form of Abl, Src or Fyn.

17. A tyrosine kinase inhibitor protein or functional equivalent thereof according to claim 16 which inhibits an oncogenic form of Abl.

18. A tyrosine kinase inhibitor protein or functional equivalent thereof according to claim 17 which inhibits BCR-Abl.

19. A fusion protein comprising a tyrosine kinase inhibitor protein or a functional equivalent thereof according to claim 1 fused to a marker domain.

20. A fusion protein according to claim 19 wherein the marker domain is green fluorescent protein.

21. An antibody that which binds to a tyrosine kinase inhibitor protein or a functional equivalent thereof according to claim 1.

22. A nucleic acid molecule encoding a tyrosine kinase inhibitor protein or a functional equivalent thereof according to claim 1.

23. An antisense nucleic acid molecule which binds under high stringency conditions to a nucleic acid molecule according to claim 22.

24. A vector comprising a nucleic acid molecule according to claim 22.

25. A host cell transformed or transfected with a nucleic acid molecule according to claim 22.

26. A method for preparing a tyrosine kinase inhibitor protein or a functional equivalent thereof comprising culturing a host cell containing a nucleic acid molecule according to claim 22 under conditions whereby said protein is expressed and recovering said protein thus produced.

27. A method of identifying an activator compounds that inhibits autoinhibition of c-Abl by the cap region comprising contacting c-Abl with a candidate activator compound and assessing whether binding between the cap region of c-Abl and the catalytic and SH2 and/or SH3 domains of c-Abl has been inhibited.

28. An activator compound identified or identifiable by the method of claim 27.

29. A method for identifying a modulator compound that restores autoinhibition of c-Abl by the cap region comprising contacting c-Abl and an activator compound according to claim 28 with a candidate modulator compound and assessing whether binding between the cap region of c-Abl and the catalytic and SH2 and/or SH3 domains of c-Abl is restored.

30. A modulator compound identified or identifiable by the method of claim 29.

31. A method of modulating the activity of a protein tyrosine kinase comprising providing a cell with an active agent said active agent selected from the group consisting of
(i) a tyrosine kinase inhibitor protein or a functional equivalent thereof according to claim
(ii) a fusion protein comprising said tyrosine kinase inhibitor protein or a functional equivalent thereof fused to a maker domain;
(iii) a nucleic acid molecule encoding said tyrosine kinase inhibitor protein or a functional equivalent thereof;
(iv) a nucleic acid molecule encoding said fusion protein;
(v) an antisense molecule which binds under high stringency conditions to a nucleic acid molecule of (iii);
(vi) an antisense molecule which binds under high stringency conditions to a nucleic acid molecule of (iv);
(vii) an activator compound that inhibits autoinhibition of c-Abl by the cap region; and
(viii) a modulation compound that restores autoinhibition of c-Abl by the cap region.

32. Use of a cap region of a c-Abl protein or a functional equivalent thereof as a tyrosine kinase inhibitor.

33. An active agent for use as a pharmaceutical said active agent selected from the group consisting of:
(i) a tyrosine kinase inhibitor protein or a functional equivalent thereof according to claim 1;
(ii) a fusion protein comprising said tyrosine kinase inhibitor protein or a functional equivalent thereof fused to a maker domain;
(iii) a nucleic acid molecule encoding said tyrosine kinase inhibitor protein or a functional equivalent thereof;
(iv) a nucleic acid molecule encoding said fusion protein;
(v) an antisense molecule which binds under high stringency conditions to a nucleic acid molecule of (iii);
(vi) an antisense molecule which binds under high stringency conditions to a nucleic acid molecule of (iv);
(vii) an activator compound that inhibits autoinhibition of c-Abl by the cap region; and
(viii) a modulation compound that restores autoinhibition of c-Abl by the cap region.

34. A pharmaceutical composition comprising an active agent in conjunction with a pharmaceutically-acceptable carrier molecule, said active agent selected from the group consisting of:
(i) a tyrosine kinase inhibitor protein or a functional equivalent thereof according to claim 1;
(ii) a fusion protein comprising said tyrosine kinase inhibitor protein or a functional equivalent thereof fused to a maker domain;
(iii) a nucleic acid molecule encoding said tyrosine kinase inhibitor protein or a functional equivalent thereof;
(iv) a nucleic acid molecule encoding said fusion protein;
(v) an antisense molecule which binds under high stringency conditions to a nucleic acid molecule of (iii);
(vi) an antisense molecule which binds under high stringency conditions to a nucleic acid molecule of (iv);
(vii) an activator compound that inhibits autoinhibition of c-Abl by the cap region; and
(viii) a modulation compound that restores autoinhibition of c-Abl by the cap region.

35. Use of an active agent in the manufacture of a medicament for the treatment of a disease with aberrant tyrosine kinase activity, said agent selected from the group consisting of:
(i) a tyrosine kinase inhibitor protein or a functional equivalent thereof according to claim 1;
(ii) a fusion protein comprising said tyrosine kinase inhibitor protein or a functional equivalent thereof fused to a maker domain;
(iii) a nucleic acid molecule encoding said tyrosine kinase inhibitor protein or a functional equivalent thereof;
(iv) a nucleic acid molecule encoding said fusion protein;
(v) an antisense molecule which binds under high stringency conditions to a nucleic acid molecule of (iii);
(vi) an antisense molecule which binds under high stringency conditions to a nucleic acid molecule of (iv);
(vii) an activator compound that inhibits autoinhibition of c-Abl by the cap region; and
(viii) a modulation compound that restores autoinhibition of c-Abl by the cap region.

36. A method of treating a disease associated with aberrant tyrosine kinase activity in a patient, comprising administering to the patient an active agent selected from the group consisting of:
(i) a tyrosine kinase inhibitor protein or a functional equivalent thereof according to claim 1;
(ii) a fusion protein comprising said tyrosine kinase inhibitor protein or a functional equivalent thereof fused to a maker domain;
(iii) a nucleic acid molecule encoding said tyrosine kinase inhibitor protein or a functional equivalent thereof;
(iv) a nucleic acid molecule encoding said fusion protein;
(v) an antisense molecule which binds under high stringency conditions to a nucleic acid molecule of (iii);
(vi) an antisense molecule which binds under high stringency conditions to a nucleic acid molecule of (iv);
(vii) an activator compound that inhibits autoinhibition of c-Abl by the cap region;
(viii) a modulation compound that restores autoinhibition of c-Abl by the cap region; and
(xi) a pharmaceutical composition comprising (ii), (iii), (iv), (v), (vi), (vii) or (viii).

37. The method according to claim 36 wherein said disease is a neurological disease or cancer.

38. The method according to claim 37 wherein said disease is leukaemia.

39. A method of diagnosing a conditions associated with an aberrant activity of a tyrosine kinase protein comprising measuring the level of an aberrant tyrosine kinase protein in a cell sample obtained from a patient using a tyrosine kinase inhibitor protein according to claim 1.

40. A method of diagnosing a condition associated with an aberrant tyrosine kinase activity of c-Abl protein comprising using a nucleic acid molecule according to claim 22 to screen for mutations in the cap region.

41. A transgenic animal comprising a nucleic acid molecule according to claim 22.

42. A c-Abl protein comprising a protease cleavage site located near the boundary of the cap region and the SH3 domain.

43. A c-Abl protein according to claim 42 wherein said protease cleavage site is a TEV protease cleavage site.

44. A fusion protein comprising a c-Abl protein according to claim 42 fused to a marker domain.

45. A method for activating the tyrosine kinase activity of c-Abl comprising supplying a cell with a c-Abl protein comprising a protease cleavage site according to claim 42 and supplying the cell with a protease to cleave at the protease cleavage site.

46. A method for producing an activated c-Abl protein comprising cleaving a c-Abl protein according to claim 42 with a protease and isolating the cleaved C-terminal region of said c-Abl protein.

47. A method for producing a tyrosine kinase inhibitor protein comprising cleaving a c-Abl protein according to claim 42 with a protease and isolating the cleaved N-terminal cap region of said c-Abl protein.

48. A nucleic acid molecule encoding a c-Abl protein according to claim 42.

49. A method for activating a c-Abl protein comprising introducing a nucleic acid molecule according to claim 48 into a cell under conditions in which it is expressed and supplying said cell with a protease.

50. A method for producing an activated c-Abl protein comprising introducing a nucleic acid molecule according to claim 48 into a cell under conditions in which it is expressed, supplying said cell with a protease and isolating the cleaved C-terminal region of said c-Abl protein

51. A method for producing a tyrosine kinase inhibitor protein comprising introducing a nucleic acid molecule according to claim 48 into a cell under conditions in which it is expressed, supplying said cell with a protease and isolating the cleaved N-terminal cap region of said c-Abl protein.

52. A transgenic animal comprising a nucleic acid molecule according to claim 48.

53. A method for activating tyrosine kinase activity of c-Abl in vivo comprising supplying a transgenic animal according to claim 52 with a protease.

54. A method for screening for a compound that restores autoinhibition of c-Abl in vivo comprising supplying a transgenic animal according to claim 52 with a protease to activate the tyrosine kinase activity of c-Abl, supplying the transgenic animal with a candidate compound and assessing the effect of the candidate compound on the tyrosine kinase activity in the cells of said transgenic animal.

55. An antibody that binds to a fusion protein according to claim 19.

56. A nucleic acid molecule encoding a fusion protein according to claim 19.

57. An antisense nucleic acid which binds under high stringency conditions to a nucleic acid molecule according to claim 56.

58. A vector comprising a nucleic acid molecule according to claim 23.

59. A vector comprising a nucleic acid molecule according to claim 56.

60. A vector comprising a nucleic acid molecule according to claim 57.

61. A host cell transformed or transfected with nucleic acid molecule according to claim 23.

62. A host cell transformed or transfected with nucleic acid molecule according to claim 56.

63. A host cell transformed or transfected with nucleic acid molecule according to claim 57.

64. A host cell transformed or transfected with a vector according to claim 24.

65. A host cell transformed or transfected with a vector according to claim 58.

66. A host cell transformed or transfected with a vector according to claim 59.

67. A host cell transformed or transfected with a vector according to claim 60.

68. A method for preparing a tyrosine kinase inhibitor protein or a functional equivalent thereof comprising culturing a host cell containing a nucleic acid molecule according to claim 56 under conditions whereby said protein is expressed and recovering said protein thus produced.

69. A method of diagnosing a condition associated with an aberrant tyrosine kinase activity of c-Abl protein comprising using a nucleic acid molecule according to claim 22 to screen for mutations in the cap region.

70. A method for activating the tyrosine kinase activity of c-Abl comprising supplying a cell with a c-Abl protein comprising a protease cleavage site according to claim 44 or a fusion protein and supplying the cell with a protease to cleave at the protease cleavage site.

71. A method for producing an activated c-Abl protein comprising cleaving a c-Abl protein according to a fusion protein according to claim 44 with a protease and isolating the cleaved C-terminal region of said c-Abl protein.

72. A method for producing a tyrosine kinase inhibitor protein comprising cleaving a c-Abl protein according to a fusion protein according to claim 44 with a protease and isolating the cleaved N-terminal cap region of said c-Abl protein.

73. A nucleic acid molecule encoding a c-Abl protein or a fusion protein according to claim 44.
